(19) United States
(12) Patent Application Publication
(10) Pub. No.: US 2022/0313722 A1
(43) Pub. Date: Oct. 6, 2022

(54) METHODS OF RESTORING LYSOSOMAL FUNCTION OF RETINAL PIGMENT EPITHELIAL CELLS BY ACTIVATION OF TFEB

(71) Applicant: University of Pittsburgh - Of the Commonwealth System of Higher Education, Pittsburgh, PA (US)

(72) Inventors: Debasish Sinha, Clarksville, MD (US); Leah Caroline Thomas Byrne, Pittsburgh, PA (US); Nadezda Anatolyena Stepicheva, Wilkinsburgh, PA (US); Sayan Ghosh, Pittsburgh, PA (US); Stacey Hose, Clear Spring, MD (US)

(73) Assignee: University of Pittsburgh - Of the Commonwealth System of Higher Education, Pittsburgh, PA (US)

(21) Appl. No.: 17/640,422

(22) PCT Filed: Mar. 6, 2020
(86) PCT No.: PCT/US20/21540

§ 371 Date: Mar. 4, 2022

Related U.S. Application Data
(60) Provisional application No. 62/897,800, filed on Sep. 9, 2019.

(51) Int. Cl. A61K 31/7088; A61P 27/00

(52) U.S. Cl. CPC A61K 31/7088; A61P 27/00

(57) ABSTRACT

A method of restoring lysosomal function of retinal pigment epithelial (RPE) cells and a method of preventing and/or treating age-related macular degeneration (AMD), Stargardt's macular retinal degeneration, neurodegenerative disease, or diabetic retinopathy in a subject are provided. The methods comprise administering (i) a nucleic acid encoding a polypeptide comprising a constitutively active form of transcription factor EB (TFEB) or (ii) the polypeptide to a subject in need thereof. Associated polypeptides, nucleic acids, vectors, and compositions thereof also are provided.

### CROSS-REFERENCE TO RELATED APPLICATIONS

This patent application claims the benefit of U.S. Provisional Patent Application No. 62/897,800 filed Sep. 9, 2019, which is incorporated by reference.

### SEQUENCE LISTING

Incorporated by reference in its entirety herein is a nucleotide/amino acid sequence listing submitted concurrently herewith.

### BACKGROUND OF THE INVENTION

Age-related macular degeneration (AMD) is the world's leading cause of blindness among the elderly. It is projected that the number of people with AMD worldwide will be 196 million in 2020, increasing to 288 million in 2040. More than 15 million Americans are affected by AMD, and the costs of treatment are in excess of $350 billion. The most recent data suggest that more than 3 million additional people in the United States will be diagnosed with the disease by 2020.

The vast majority of patients suffer from atrophic (dry) AMD. However, due to the relentless progression of the disease, within 10 years they will develop advanced disease. The burden of dry AMD is increasing as the “baby boomers” age. Despite this growing population of afflicted individuals, no definitive treatment (other than the AREDS II formulation for intermediate AMD) or prevention for dry AMD is currently available. The dry type of AMD affects approximately 80-90% of individuals with the disease. Its cause is unknown, and it usually progresses more slowly than the wet type.

There is currently anti-VEGF therapy available for patients suffering from wet AMD, such as AVASTIN™ (bevacizumab), LUCENTIS™ (ranibizumab injection), and EYLEA™ (aflibercept). However, the treatment regime (requires injections every month) is very expensive, does not work long-term, and does not help all patients.

Due to the large number of patients with atrophic AMD, there is a desire for a new treatment that targets early disease at a stage before vision is lost.

### BRIEF SUMMARY OF THE INVENTION

The invention provides a method of restoring lysosomal function of retinal pigment epithelial (RPE) cells comprising administering (i) a nucleic acid encoding a polypeptide comprising a constitutively active form of transcription factor EB (TFEB) or (ii) the polypeptide to a subject in need thereof. The nucleic acid can be comprised in a vector (e.g., AAV vector)

The subject (e.g., human) can have age-related macular degeneration (AMD) or be at risk for developing AMD, wherein the AMD can be wet AMD or atrophic (dry) AMD, which may include geographic atrophy. The subject can have Stargardt's macular retinal degeneration or be at risk for developing Stargardt's macular retinal degeneration. The subject can have a neurodegenerative disease or be at risk for developing a neurodegenerative disease, such as Alzheimer's disease or Parkinson's disease. Alternatively or additionally, the subject can have diabetic retinopathy (DR) or be at risk for developing DR.

As such, the invention provides a method of preventing and/or treating AMD, Stargardt's macular retinal degeneration, neurodegenerative disease, or diabetic retinopathy in a subject comprising administering (i) a nucleic acid encoding a polypeptide comprising a constitutively active form of transcription factor EB (TFEB) or (ii) the polypeptide to the subject. The nucleic acid can be comprised in a vector (e.g., AAV vector).

The polypeptide can comprise the amino acid sequence of SEQ ID NO: 1, except that the serine at one or more of positions 138, 142, 211, 455, 462, 463, 466, 467, and 469 of SEQ ID NO: 1 is, independently, substituted with another amino acid. In particular,

(i) the serine at position 138 of SEQ ID NO: 1 can be substituted with an alanine, and/or

(ii) the serine at position 142 of SEQ ID NO: 1 can be substituted with an alanine, and/or

(iii) the serine at position 211 of SEQ ID NO: 1 can be substituted with an alanine, and/or

(iv) the serine at position 455 of SEQ ID NO: 1 can be substituted with an alanine, and/or

(v) the serine at position 462 of SEQ ID NO: 1 can be substituted with an aspartic acid, and/or

(vi) the serine at position 463 of SEQ ID NO: 1 can be substituted with an aspartic acid, and/or

(vii) the serine at position 466 of SEQ ID NO: 1 is substituted with an aspartic acid, and/or

(viii) the serine at position 467 of SEQ ID NO: 1 is substituted with an alanine, and/or

(ix) the serine at position 469 of SEQ ID NO: 1 is substituted with an aspartic acid.

In one embodiment, the polypeptide can comprise the amino acid sequence of SEQ ID NO: 1, except that the serine at each one of positions 138, 142, and 211 of SEQ ID NO: 1 is respectively substituted with an alanine.

In another embodiment, the polypeptide can comprise the amino acid sequence of SEQ ID NO: 1, except that the serine at position 455 of SEQ ID NO: 1 is substituted with an alanine and, optionally, the serine at position 211 of SEQ ID NO: 1 is substituted with an alanine.

In a further embodiment, the polypeptide can comprise the amino acid sequence of SEQ ID NO: 1, except that the serine at each one of positions 462, 463, 466, and 469 of SEQ ID NO: 1 is respectively substituted with an aspartic acid and the serine at position 467 of SEQ ID NO: 1 is substituted with an alanine.

The invention also provides (a) a pharmaceutically-acceptable carrier and (b) the polypeptide, a nucleic acid encoding the polypeptide, or a vector comprising the nucleic acid.

The invention further relates to a pharmaceutical composition for preventing and/or treating AMD (e.g., atrophic AMD or wet AMD), Stargardt's macular retinal degeneration, neurodegenerative disease (e.g., Alzheimer's disease or Parkinson's disease), or diabetic retinopathy comprising (i) a nucleic acid encoding a polypeptide comprising a constitutively active form of TFEB or (ii) the polypeptide.

The invention relates to the use of (i) a nucleic acid encoding a polypeptide comprising a constitutively active form of TFEB or (ii) the polypeptide for the manufacture of a pharmaceutical composition for preventing and/or treating AMD (e.g., atrophic AMD or wet AMD), Stargardt's macular retinal degeneration, neurodegenerative disease (e.g., Alzheimer's disease or Parkinson's disease), or diabetic retinopathy.

The invention relates to the use of (i) a nucleic acid encoding a polypeptide comprising a constitutively active form of TFEB or (ii) the polypeptide for preventing and/or treating AMD, (e.g., atrophic AMD or wet AMD), Stargardt's macular retinal degeneration, neurodegenerative disease (e.g., Alzheimer's disease or Parkinson's disease), or diabetic retinopathy.

The invention also relates to (i) a nucleic acid encoding a polypeptide comprising a constitutively active form of TFEB or (ii) the polypeptide for use in preventing and/or treating AMD (e.g., atrophic AMD or wet AMD), Stargardt's macular retinal degeneration, neurodegenerative disease (e.g., Alzheimer's disease or Parkinson's disease), or diabetic retinopathy.

### BRIEF DESCRIPTION OF THE SEVERAL VIEWS OF THE DRAWINGS

FIGS. 1A-1C show TFEB immunolabeling in the RPE/choroid of a 78 year old male control (FIG. 1A) and 84 year old male with atrophic AMD (FIG. 1B). In the control eye, the intense TFEB immunoreactivity is prominent in RPE nuclei (arrows), but is absent in the RPE nuclei of the AMD eye (asterisk) in (FIG. 1B), as depicted in the graph (FIG. 1C). Note: multilayered RPE in (B). Bar=20 μm; *P<0.05.

FIGS. 2A-2B demonstrate that expression of proteins in the endocytic pathway is reduced in RPE cells from Cryba1 conditional knockout (cKO) mice (Cryba1 deleted specifically from RPE cells as described in Valapala et al., Nat. Commun., 4: 1629 (2013)). FIG. 2A shows that Rab5, an early endosome marker, is reduced in 5-month-old Cryba1 cKO RPE cells compared to age-matched floxed control (Cryba1^{fl/fl} as described in Valapala et al., Sci. Rep., 5: 8755 (2015)) RPE cells as shown by western blot (WB). Actin was used as internal control. The graph shows the mean±SD and *P<0.05 (two-tail t test), n=5. FIG. 2B shows that EEA1, a protein that is required for fusion of early and late endosomes and for sorting at the early endosome level, and APPL1 that regulates vesicle trafficking and endosome signaling, are reduced in 5-month-old Cryba1 cKO RPE cells compared to Cryba1^{fl/fl} RPE cells as shown by WB. Actin was used as internal control.

FIGS. 3A-3C demonstrate that Cryba1 cKO mice show RPE cell heterogeneity. FIG. 3A shows immunostaining of EBP50 on retina sections of 1-month and 9-month old cKO mice, indicating reduced EBP50 and microvilli length in RPE cells compared to controls. FIG. 3B shows RPE extracts of 3-month old mice had decreased expression of apical side proteins (ERM and EBP50) in cKO RPE cells. FIG. 3C is RPE flat mounts of 4-month old Cryba1 cKO mice showing RPE heterogeneity as visualized using Phalloidin (F-actin) and EBP50 (microvilli) antibody. Both F-actin and EBP50 staining show areas of cell loss at the apical side (arrows) in cKO RPE. The pockets of cell loss are severe as the mice age (not shown). Orthogonal image (merge/Ortho) shows dropout of RPE microvilli (arrows). Bar=20 mm. The graph shows Mean±S.D and *P<0.05, **P<0.01 (two-tail t test), n=5.

FIG. 4 shows altered clathrin mediated endocytosis in RPE cells from Cryba1 cKO mice. Live cell imaging of clathrin mediated endocytosis from the apical side of floxed control and cKO RPE cells was performed with Total Internal Reflection Fluorescence (TIRF) microscopy. The RPE-Choroid-Sclera complex was cultured in glass-bottom dishes and infected with rAVCMV-LifeAct-TagGFP2 and Ad-CMV-FAP-mClta to visualize actin and clathrin, respectively. TIRF images and/or videos of the flat mounts were taken on an inverted Nikon Ti with a Plan Apo TIRF 1.49 NA 100× oil objective and Prime 95b camera (Photometrics). Over 12 seconds, clathrin vesicles fail to enter the apical plasma membrane (arrows), while insertion occurs in floxed controls.

FIG. 5 is a schematic diagram for WT-TFEB and constitutively active TFEB-S210A preparation described in the Example.

FIGS. 6A-6C demonstrate that AAV2-TFEB vector is suitable for transfection in RPE cells. RPE/choroid (A) from an uninfected mouse and (B) following subretinal injection of AAV2-TFEB-GFP demonstrate efficient infection of more than 85-90% of RPE cells after a single subretinal infection. No GFP-expressing cells were found in the neurosensory retina or choroid (not shown). (C) Peak expression is observed after 7 days. Again, no GFP-expressing cells were found in the neurosensory retina or choroid for two weeks following infection (not shown).

FIGS. 7A-7C are graphics demonstrating that rejuvenating TFEB function mitigates alterations in lysosomal function and autophagy in RPE cells of Cryba1 KO mice. FIG. 7A details that administration of an AAV vector expressing a constitutively active mouse TFEB (TFEB^{S210A}) to RPE cells of Cryba1 KO mice results in translocation of TFEB^{S210A} to the nucleus of the RPE cells. FIG. 7B is a graph showing the relative expression of CTSB, LAMP2, ATP6VOA1, and CTSD in RPE cells of Cryba1 KO mice infected with the AAV vector expressing TFEB^{S210A} (4) as compared to wild-type mice (1), Cryba1 KO mice without vector (2), and Cryba1 KO mice administered the AAV vector expressing wild-type TFEB (3). FIG. 7C demonstrates the relative expression of p62/SQSTM1 in RPE cells of Cryba1 KO mice infected with the AAV vector expressing TFEB^{S210A} as compared to expression in RPE cells of Cryba1 KO mice infected with AAV vector expressing wild-type TFEB. Empty AAV had no effect. Mean±S.D., n=3. P-values were evaluated by one-way AVOVA and Tukey's post-hoc test and are representative of at least 3 independent experiments. *P<0.05 and **P<0.01.

FIGS. 8A-8C demonstrate iron accumulation and inflammasome activation in RPE cells of aged Cryba1 cKO mice. FIG. 8A shows that iron overload due to a lysosomal abnormality in RPE cells may cause inflammasome activation and cell death. FIG. 8B shows increased redox sensitive ferrous ion levels in RPE cells of 4 mo (3, 4) or 10 mo (7, 8) aged Cryba1 cKO mice compared to 4 mo (1, 2) or 10 mo (5, 6) aged Cryba1^{fl/fl} mice (controls) under fed and starved conditions. FIG. 8C demonstrates elevated levels of inflammasome markers NLRP3 and cleaved caspase-1 in RPE cells of aged Cryba1 cKO mice relative to age-matched controls.

FIGS. 9A-9C demonstrate that TFEB activation assuages iron accumulation and inflammasome activation in Cryba1 KO RPE cells in vitro. FIG. 9A shows the administration of an AAV vector expressing a constitutively active mouse TFEB (TFEB^{S210A}) to Cryba1 KO RPE cells in the presence of iron chelator Lipocalin-2 (LCN-2) and an iron source ferric ammonium citrate (FAC). FIGS. 9B-9C show that Cryba1 KO RPE cells infected with an AAV vector expressing TFEB^{S210A} could reverse iron accumulation and inflammasome activation (increased NLRP3 and IL-β expression) compared to Cryba1 KO RPE cells infected with AAV vector expressing wild-type TFEB. *P<0.05 and **P<0.01.

FIGS. 10A-10B demonstrate that rejuvenating TFEB function mitigates alterations in lysosomal function and autophagy in RPE cells of Cryba1 cKO mice. 9 month old Cryba1 cKO mice were sub-retinally injected with an AAV vector expressing WT TFEB (WT TFEB vector) or an AAV vector expressing a constitutively active mouse TFEB (TFEB^{S210A}) (mutant TFEB vector). FIG. 10A shows that administration of the mutant vector to RPE cells of Cryba1 cKO mice results in the rejuvenation of CLEAR (coordinated lysosomal expression and regulation) network genes by qPCR. FIG. 10B demonstrates that administration of the mutant vector to Cryba1 cKO mice resulted in improved autophagosome clearance with reduced cellular p62/SQSTM1 levels in RPE cells Animals were sacrificed two months later, and the RPE-choroid complex was harvested. *P<0.05, **P<0.01 (n=4).

FIGS. 11A-11B demonstrate predominantly cytosolic sequestration of TFEB in RPE cells from fasted and fed Cryba1 KO mice (FIG. 11A) concomitant with a decrease in CLEAR network genes in RPE cells from Cryba1 cKO mice (FIG. 11B), relative to Cryba1^{fl/fl} control.

FIGS. 12A-12B demonstrate the lysosomal pH in RPE cells of Cryba1 cKO mice relative to Cryba1^{fl/fl} control. FIG. 12A is a graph showing a significant increase in lysosomal pH in RPE cells of Cryba1 cKO mice relative to control. FIG. 12B is a graph demonstrating that overexpressing Cryba1 (by administration of pCDNA-Cryba1) in cKO RPE cells in vitro rescues the pH abnormality.

FIGS. 13A-13B demonstrate cathepsin D enzyme activity in RPE cells of Cryba1 cKO mice relative to Cryba1^{fl/fl} control. FIG. 13A is a graph showing significant reduction of cathepsin D enzyme activity in RPE cells of Cryba1 cKO mice relative to Cryba1^{fl/fl} control. FIG. 13B is a graph demonstrating that overexpressing Cryba1 (by administration of pCDNA-Cryba1) in cKO RPE cells in vitro rescues cathepsin D enzyme activity.

FIG. 14A-14C demonstrate abnormalities in Cryba1 knockout mice. FIG. 14A shows localization of βA3/A1-crystallin in the lysosomal lumen fraction (fraction 1), with minimal expression in the lysosomal membrane fraction (fraction 2). FIG. 14B demonstrates Cryba1 (encoding for βA3/A1-crystallin) conditional (deleted specifically from RPE) and knockout mice (Cryba1 cKO). FIG. 14C shows a schematic representation of the development of the RPE abnormality and AMD-like phenotype in Cryba1 cKO mice from 2-3 weeks of age and up to 9 months.

FIGS. 15A-15B show autophagosome and autophagolysosome formation in Cryba1 cKO RPE cells. FIG. 15A shows live cell imaging of RPE cells isolated from WT or cKO mice and transfected with pH-sensitive reporter (mCherry-GFP-LC3) showing a decrease in autophagolysosome formation in Cryba1 cKO RPE cells relative to controls, which was rescued upon Cryba1 overexpression. FIG. 17B shows quantification of the number of autophagosome and autophagolysosome puncta under different conditions.

FIG. 16A-16B demonstrate cathepsin D levels in WT and Cryba1 KO RPE cells Immunofluorescence (FIG. 16A) and western blot (FIG. 16B) showing a decrease in Cathepsin D levels in the RPE cells from Cryba1 KO mice compared to age-matched controls.

FIGS. 17A-17B demonstrate that Cryba1 KO mice show an age-related macular degeneration-like phenotype. FIG. 17A is an immunofluorescence image of RPE flat mounts showing loss of the cobblestone-like structure (arrows) of RPE cells along with noticeable decrease in the expression of RPE65 (a known marker of RPE cells). FIG. 17B is electron microscopy images showing accumulation of large vacuoles (arrows) and basal lamina deposits (asterisks and inset) in the RPE cells of Cryba1 KO mice with increasing age.

FIGS. 18A-C show that the RPE of the Cryba1 cKO mouse retains (auto) phagosomes. Electron microscopy images showing accumulation of large autophagosomes (arrows in FIGS. 18A-B), which are laden with photoreceptor outer segments and not being degraded (arrow in FIG. 18C).

### DETAILED DESCRIPTION OF THE INVENTION

The inventors discovered that the active form of TFEB could rejuvenate the function in RPE cells and rescue an AMD-like phenotype. This concept is significant beyond AMD, and provides unique insights into aging, and age-related and neurodegenerative diseases where lysosomal dysfunction and cellular heterogeneity are important pathophysiologic features.

The invention provides a method of restoring lysosomal function of retinal pigment epithelial (RPE) cells comprising administering (i) a nucleic acid encoding a polypeptide comprising a constitutively active form of TFEB or (ii) the polypeptide to a subject in need thereof. The invention also provides related polypeptides, nucleic acids, vectors, and compositions.

The subject refers to humans or other animals (e.g., a mammal, such as a mouse, rat, guinea pig, hamster, cat, dog, rabbit, pig, cow, horse, or primate), wherein the subject can refer to animals (e.g., humans) in need of prevention or treatment of a disease.

In one embodiment, the subject has age-related macular degeneration (AMD) or is at risk for developing AMD. The AMD can be wet AMD or atrophic (dry) AMD. The subject may have geographic atrophy secondary to AMD. As such, the invention also provides a method of treating and/or preventing AMD in the subject.

In one embodiment, the subject has Stargardt's macular retinal degeneration or is at risk for developing Stargardt's macular retinal degeneration. As such, the invention also provides a method of treating and/or preventing Stargardt's macular retinal degeneration.

In another embodiment, the subject has a neurodegenerative disease or is at risk for developing a neurodegenerative disease, such as Alzheimer's disease or Parkinson's disease. As such, the invention also provides a method of treating and/or preventing a neurodegenerative disease, such as Alzheimer's disease or Parkinson's disease, in the subject.

In a further embodiment, the subject has diabetic retinopathy (DR) or is at risk for developing DR. The subject also may have diabetic macular edema (DME) or be at risk for developing DME. As such, the invention also provides a method of treating and/or preventing DR or DME in the subject.

As such, the invention provides a method of treating and/or preventing AMD, Stargardt's macular retinal degeneration, neurodegenerative disease, or diabetic retinopathy in a subject comprising administering (i) a nucleic acid encoding a polypeptide comprising a constitutively active form of TFEB or (ii) the polypeptide to the subject.

The polypeptide can comprise, consist essentially of, or consist of the amino acid sequence of SEQ ID NO: 1, except that the serine at one or more (e.g., two, three, four, five six, seven, eight, or nine) of positions 138, 142, 211, 455, 462, 463, 466, 467, and 469 of SEQ ID NO: 1 is, independently, substituted with another amino acid (i.e., a naturally or non-naturally occurring amino acid other than serine). For example:

(i) the serine at position 138 of SEQ ID NO: 1 can be substituted with an alanine, and/or

(ii) the serine at position 142 of SEQ ID NO: 1 can be substituted with an alanine, and/or

(iii) the serine at position 211 of SEQ ID NO: 1 can be substituted with an alanine, and/or

(iv) the serine at position 455 of SEQ ID NO: 1 can be substituted with an alanine, and/or

(v) the serine at position 462 of SEQ ID NO: 1 can be substituted with an aspartic acid, and/or

(vi) the serine at position 463 of SEQ ID NO: 1 can be substituted with an aspartic acid, and/or

(vii) the serine at position 466 of SEQ ID NO: 1 is substituted with an aspartic acid, and/or

(viii) the serine at position 467 of SEQ ID NO: 1 is substituted with an alanine, and/or

(ix) the serine at position 469 of SEQ ID NO: 1 is substituted with an aspartic acid.

In one embodiment, the polypeptide comprises, consists essentially of, or consists of the amino acid sequence of SEQ ID NO: 1, except that the serine at each one of positions 138, 142, and 211 of SEQ ID NO: 1 is respectively substituted with an alanine.

In another embodiment, the polypeptide comprises, consists essentially of, or consists of the amino acid sequence of SEQ ID NO: 1, except that the serine at position 455 of SEQ ID NO: 1 is substituted with an alanine and, optionally, the serine at position 211 of SEQ ID NO: 1 is substituted with an alanine. Thus, the invention provides both (i) a polypeptide comprising the amino acid sequence of SEQ ID NO: 1, except that the serine at position 455 of SEQ ID NO: 1 is substituted with an alanine, and (ii) a polypeptide comprising the amino acid sequence of SEQ ID NO: 1, except that the serine at each of positions 211 and 455 of SEQ ID NO: 1 is respectively substituted with an alanine.

In a further embodiment, the polypeptide comprises, consists essentially of, or consists of the amino acid sequence of SEQ ID NO: 1, except that the serine at each one of positions 462, 463, 466, and 469 of SEQ ID NO: 1 is respectively substituted with an aspartic acid and the serine at position 467 of SEQ ID NO: 1 is substituted with an alanine.

The polypeptide can be prepared by any of a number of conventional techniques. In this respect, the polypeptide sequence can be synthetic, recombinant, isolated, and/or purified.

The polypeptide can be isolated or purified from a recombinant source. For instance, a DNA fragment encoding a desired polypeptide can be subcloned into an appropriate vector using well-known molecular genetic techniques. The fragment can be transcribed and the polypeptide subsequently translated in vitro. Commercially available kits also can be employed. The polymerase chain reaction optionally can be employed in the manipulation of nucleic acids.

The polypeptide also can be synthesized using an automated peptide synthesizer in accordance with methods known in the art. Alternately, the polypeptide can be synthesized using standard peptide synthesizing techniques well-known to those of skill in the art. In particular, the polypeptide can be synthesized using the procedure of solid-phase synthesis. If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The protein-containing mixture then can be extracted, for instance, with diethyl ether, to remove non-peptidic organic compounds, and the synthesized polypeptide can be extracted from the resin powder (e.g., with about 25% w/v acetic acid). Following the synthesis of the polypeptide, further purification (e.g., using HPLC) optionally can be performed in order to eliminate any incomplete proteins, polypeptides, peptides or free amino acids. Amino acid and/or HPLC analysis can be performed on the synthesized polypeptide to validate its identity. For other applications according to the invention, it may be preferable to produce the polypeptide as part of a larger fusion protein, either by chemical conjugation or through genetic means, such as are known to those skilled in the art. In this regard, the invention also provides a fusion protein comprising the polypeptide and one or more other protein(s) having any desired properties or functions, such as to facilitate isolation, purification, analysis, or stability of the fusion protein.

The invention also provides a nucleic acid encoding the polypeptide. “Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acid is recombinant As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acid (e.g., DNA, RNA, cDNA, and the like) can be produced in any suitable matter including, but not limited to recombinant production and commercial synthesis. In this respect, the nucleic acid sequence can be synthetic, recombinant, isolated, and/or purified.

The nucleic acid can be constructed based on chemical synthesis and/or enzymatic ligation reactions using procedures known in the art. See, for example, Green et al. (eds.), Molecular Cloning, A Laboratory Manual, 4^{th} Edition, Cold Spring Harbor Laboratory Press, New York (2012). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid encoding the polypeptide can be provided as part of a construct comprising the nucleic acid and elements that enable delivery of the nucleic acid to a cell, and/or expression of the nucleic acid in a cell. For example, the polynucleotide sequence encoding the polypeptide can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers (e.g., CMV enhancer), transcription terminators, a start codon (i.e., ATG) in front of a protein-encoding gene, splicing signal for introns, maintenance of the correct reading frame of that gene to permit proper translation of mRNA, and stop codons. Suitable promoters include, but are not limited to, a hVMD2 promoter, an SV40 early promoter, RSV promoter, adenovirus major late promoter, human CMV immediate early I promoter, poxvirus promoter, 30K promoter, I3 promoter, sE/L promoter, 7.5K promoter, 40K promoter, andC1 promoter.

A nucleic acid encoding the polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the polypeptide can be isolated by polymerase chain reaction of cDNA using primers based on the DNA sequence of the molecule. A wide variety of cloning and in vitro amplification methodologies are well known to persons skilled in the art.

The invention provides a vector comprising the nucleic acid. The nucleic acid can be inserted into any suitable vector. The selection of vectors and methods to construct them are commonly known in the art and are described in general technical references.

Suitable vectors include those designed for propagation and expansion or for expression or both. Examples of suitable vectors include, for instance, plasmids, plasmid-liposome complexes, CELid vectors (see, e.g., Li et al., PLoS One, 8(8): e69879. doi: 10.1371/journal.pone.0069879 (2013)) and viral vectors, e.g., parvoviral-based vectors (i.e., AAV vectors), retroviral vectors, herpes simplex virus (HSV)-based vectors, adenovirus-based vectors, and poxvirus vectors. Any of these expression constructs can be prepared using standard recombinant DNA techniques.

In one aspect of the invention, the vector is a viral vector, such as an AAV vector. The AAV vector may be suitable for packaging into any AAV serotype or variant thereof that is suitable for administration to ocular cells (e.g., RPE cells). Examples of suitable AAV serotypes may include, but are not limited to, AAV1, AAV2, AAV3, AAV4, AAV5, AAV6, AAV7, AAV8, AAV9, variants thereof, and engineered or newly synthesized AAV viruses such as AAV.7m8.

The AAV vector may be packaged in a capsid protein, or fragment thereof, of any of the AAV serotypes described herein. Preferably, the vector is packaged in an AAV2 capsid.

A suitable recombinant AAV may be generated by culturing a packaging cell which contains a nucleic acid sequence encoding an AAV serotype capsid protein, or fragment thereof, as defined herein; a functional rep gene; any of the inventive vectors described herein; and sufficient helper functions to permit packaging of the inventive vector into the AAV capsid protein. The components required by the packaging cell to package the inventive AAV vector in an AAV capsid may be provided to the host cell in trans. Alternatively, any one or more of the required components (e.g., inventive vector, rep sequences, capsid sequences, and/or helper functions) may be provided by a stable packaging cell which has been engineered to contain one or more of the required components using methods known to those of skill in the art.

In an embodiment of the invention, the AAV vector is self-complementary. Self-complementary vectors may, advantageously, overcome the rate-limiting step of second-strand DNA synthesis and confer earlier onset and stronger gene expression.

In an embodiment of the invention, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The vector can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

In addition to the nucleic acid encoding the polypeptide, the vector can comprise one or more nucleic acid sequences encoding one or more polypeptides for delivery and expression in a host (e.g., a mammal, such as a mouse, rat, guinea pig, hamster, cat, dog, rabbit, pig, cow, horse, or primate (e.g., human))

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The vector may further comprise regulatory sequences that permit one or more of the transcription, translation, and expression of nucleic acid comprised in the vector in a cell transfected with the vector or infected with a virus that comprises the vector. As used herein, “operably linked” sequences include both regulatory sequences that are contiguous with the nucleotide sequence encoding constitutively active TFEB polypeptide and regulatory sequences that act in trans or at a distance to control the nucleotide sequence encoding constitutively active TFEB polypeptide.

The regulatory sequences may include appropriate transcription initiation, termination, promoter and enhancer sequences; RNA processing signals such as splicing and polyadenylation (polyA) signal sequences; sequences that stabilize cytoplasmic mRNA; sequences that enhance translation efficiency (i.e., Kozak consensus sequence); sequences that enhance protein stability; and when desired, sequences that enhance secretion of the encoded product. PolyA signal sequences may be synthetic or may be derived from many suitable species, including, for example, SV-40, human and bovine.

When the vector is for administration to a host (e.g., human), the vector (e.g., AAV) preferably has a low replicative efficiency in a target cell (e.g., no more than about 1 progeny per cell or, more preferably, no more than 0.1 progeny per cell are produced). Replication efficiency can readily be determined empirically by determining the virus titer after infection of the target cell.

The polypeptide, nucleic acid, or vector can be formulated as a composition (e.g., pharmaceutical composition) comprising the polypeptide, nucleic acid, or vector and a carrier (e.g., a pharmaceutically or physiologically acceptable carrier). Furthermore, the polypeptide, nucleic acid, vector, or composition of the invention can be used in the methods described herein alone or as part of a pharmaceutical formulation.

The composition (e.g., pharmaceutical composition) can comprise more than one polypeptide, nucleic acid, vector, or composition of the invention. Alternatively, or in addition, the composition can comprise one or more other pharmaceutically active agents or drugs. Examples of such other pharmaceutically active agents or drugs that may be suitable for use in the pharmaceutical composition include lampalizumab (anti-complement factor D; Genentech) for patients with geographic atrophy secondary to AMD); brolicizumab (pan-isoform ant-VEGF-A; Novartis) for wet AMD; OPT-302 (soluble VEGF-C/D receptor; Ophthea) for wet AMD and diabetic retinal edema (DME); PanOptica's topical VEGF inhibitor for wet AMD; pegpleranib (DNA aptamer binding to PDGF isoforms; Ophtotech/Novartis) optionally combined with LUCENTIS™ (ranibizumab injection); rinucumab (anti-PDGF receptor; Regeneron) optionally co-formulated with EYLEA™ (aflibercept); DE-120 (anti-PDGF/VEGF bispecific; Santen); vorolanib (oral RTK inhibitor that inhibits kinase activity for pDGF and VEGF; Tyrogenex); nevacumab (anti-angiopoeitin 2; Regeneron) optionally combined with EYLEA™ (aflibercept); RG-7716 (anti-angiopoeitin 2/VEGF bispecific; Chugai); ARP-1536 (anti VE PTP; Akebia/Aeripo); ICON-1 (chimeric protein binding to Tissue Factor; Iconic Therapeutic); and carotuximab (anti-endogin; Tracon/Santen).

The carrier can be any of those conventionally used and is limited only by physio-chemical considerations, such as solubility and lack of reactivity with the active compound(s) and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular polypeptide, nucleic acid, vector, or composition thereof of the invention and other active agents or drugs used, as well as by the particular method used to administer the polypeptide, nucleic acid, vector, or composition thereof.

The polypeptide, nucleic acid, vector, or composition thereof can be administered to the subject by any method. For example, the polypeptide, nucleic acid encoding the polypeptide, or vector comprising the nucleic acid can be introduced into a cell (e.g., in a subject) by any of various techniques, such as by contacting the cell with the nucleic acid or the vector as part of a construct, as described herein, that enables the delivery and expression of the nucleic acid. Specific protocols for introducing and expressing nucleic acids in cells are known in the art.

Any suitable dose of the polypeptide, nucleic acid, vector, or composition thereof can be administered to a subject. The appropriate dose will vary depending upon such factors as the subject's age, weight, height, sex, general medical condition, previous medical history, and disease progression, and can be determined by a clinician. The amount or dose should be sufficient to effect the desired biological response, e.g., a therapeutic or prophylactic response, in the subject over a clinically reasonable time frame.

For example, the polypeptide can be administered in a dose of about 0.05 mg to about 10 mg (e.g., 0.1 mg, 0.5 mg, 1 mg, 2 mg, 3 mg, 4 mg, 5 mg, 6 mg, 7 mg, 8 mg, 9 mg, and ranges thereof) per vaccination of the host (e.g., mammal, such as a human), and preferably about 0.1 mg to about 5 mg per vaccination. Several doses (e.g., 1, 2, 3, 4, 5, 6, or more) can be provided (e.g., over a period of weeks or months).

The dosing period may be appropriately determined depending on the therapeutic progress. In embodiments, the dosing period may comprise less than one year, less than 9 months, less than 8 months, less than 7 months, less than 6 months, less than 5 months, less than 4 months, less than 3 months, less than 2 months, or one month. In other embodiments, the dosing period may comprise three doses per day, two doses per day, or one dose per day for the length of the dosing period.

When the vector is a viral vector, a suitable dose can include about 1×10⁵ to about 1×10¹² (e.g., 1×10⁶, 1×10⁷, 1×10⁸, 1×10⁹, 1×10¹⁰, 1×10¹¹, and ranges thereof) plaque forming units (pfus), although a lower or higher dose can be administered to a host.

The polypeptide, nucleic acid, vector, or composition thereof can be administered to the subject by various routes including, but not limited to, topical, subcutaneous, intramuscular, intradermal, intraperitoneal, intrathecal, intravenous, subretinal injection, and intravitreal injection. In one embodiment, the polypeptide, nucleic acid, vector, or composition can be directly administered (e.g., locally administered) by direct injection into the eye by subretinal or inravitreal injection or by topical application (e.g., as eye drops). When multiple administrations are given, the administrations can be at one or more sites in a subject and a single dose can be administered by dividing the single dose into equal portions for administration at one, two, three, four or more sites on the individual.

Administration of the polypeptide, nucleic acid, vector, or composition thereof can be “prophylactic” or “therapeutic.” When provided prophylactically, the polypeptide, nucleic acid, vector, or composition thereof is provided in advance of a subject's diagnosis with AMD, neurodegenerative disease, DR, and/or DME. For example, subjects at risk for developing AMD, neurodegenerative disease, DR, and/or DME are a preferred group of patients treated prophylactically. The prophylactic administration of the polypeptide, nucleic acid, vector, or composition thereof prevents, ameliorates, or delays AMD, neurodegenerative disease, DR, and/or DME. When provided therapeutically, the polypeptide, nucleic acid, vector, or composition thereof is provided at or after the diagnosis of AMD, neurodegenerative disease, DR, and/or DME.

When the subject has already been diagnosed with AMD, neurodegenerative disease, DR, or DME, the polypeptide, nucleic acid, vector, or composition thereof can be administered in conjunction with other therapeutic treatments such as lampalizumab (anti-complement factor D; Genentech); brolicizumab (pan-isoform ant-VEGF-A; Novartis); OPT-302 (soluble VEGF-C/D receptor; Ophthea); PanOptica's topical VEGF inhibitor; pegpleranib (DNA aptamer binding to PDGF isoforms; Ophtotech/Novartis); LUCENTIS™ (ranibizumab injection); rinucumab (anti-PDGF receptor; Regeneron); EYLEA™ (aflibercept); DE-120 (anti-PDGF/VEGF bispecific; Santen); vorolanib (oral RTK inhibitor that inhibits kinase activity for pDGF and VEGF; Tyrogenex); nevacumab (anti-angiopoeitin 2; Regeneron); RG-7716 (anti-angiopoeitin 2/VEGF bispecific; Chugai); ARP-1536 (anti VE PTP; Akebia/Aeripo); ICON-1 (chimeric protein binding to Tissue Factor; Iconic Therapeutic); and/or carotuximab (anti-endogin; Tracon/Santen).

The terms “treat,” and “prevent,” as used herein, do not necessarily imply 100% or complete treatment or prevention. Rather, there are varying degrees of treatment or prevention of which a person of ordinary skill in the art recognizes as having a potential benefit or therapeutic effect.

There are a variety of suitable formulations of the pharmaceutical composition for the inventive methods. Any suitable carrier can be used within the context of the invention, and such carriers are well known in the art. The choice of carrier will be determined, in part, by the particular site to which the composition is to be administered (e.g., ocular cells, RPE cells, photoreceptor cells, rods, and cones) and the particular method used to administer the composition. The pharmaceutical composition can optionally be sterile or sterile with the exception of the one or more adeno-associated viral vectors.

Suitable formulations for the pharmaceutical composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the pharmaceutical composition for use in the inventive method is formulated to protect the polypeptide, nucleic acid, or vector from damage prior to administration. For example, the pharmaceutical composition can be formulated to reduce loss of the polypeptide, nucleic acid, or vector on devices used to prepare, store, or administer the polypeptide, nucleic acid, or vector, such as glassware, syringes, or needles. The pharmaceutical composition can be formulated to decrease the light sensitivity and/or temperature sensitivity of the polypeptide, nucleic acid, or vector. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition may extend the shelf life of the polypeptide, nucleic acid, or vector, facilitate administration, and increase the efficiency of the inventive method.

A pharmaceutical composition also can be formulated to enhance transduction/transfection efficiency of the polypeptide, nucleic acid, or vector. In addition, a person of ordinary skill in the art will appreciate that the pharmaceutical composition can comprise other therapeutic or biologically-active agents.

The following embodiments are exemplified:

Embodiment 1. A method of restoring lysosomal function of retinal pigment epithelial (RPE) cells comprising administering (i) a nucleic acid encoding a polypeptide comprising a constitutively active form of transcription factor EB (TFEB) or (ii) the polypeptide to a subject in need thereof.

Embodiment 2. The method of embodiment 1, wherein the subject has age-related macular degeneration (AMD) or is at risk for developing AMD.

Embodiment 3. The method of embodiment 2, wherein the AMD is atrophic AMD, optionally wherein the atrophic AMD includes geographic atrophy.

Embodiment 4. The method of embodiment 2, wherein the AMD is wet AMD.

Embodiment 5. The method of embodiment 1, wherein the subject has Stargardt's macular retinal degeneration or is at risk for developing Stargardt's macular retinal degeneration.

Embodiment 6. The method of embodiment 1, wherein the subject has a neurodegenerative disease or is at risk for developing a neurodegenerative disease.

Embodiment 7. The method of embodiment 6, wherein the neurodegenerative disease is Alzheimer's disease or Parkinson's disease.

Embodiment 8. The method of embodiment 1, wherein the subject has diabetic retinopathy (DR) or is at risk for developing DR.

Embodiment 9. A method of preventing and/or treating age-related macular degeneration (AMD), Stargardt's macular retinal degeneration, neurodegenerative disease, or diabetic retinopathy in a subject comprising administering (i) a nucleic acid encoding a polypeptide comprising a constitutively active form of transcription factor EB (TFEB) or (ii) the polypeptide to the subject.

Embodiment 10. The method of embodiment 9, wherein the AMD is atrophic AMD, optionally wherein the atrophic AMD includes geographic atrophy.

Embodiment 11. The method of embodiment 9, wherein the AMD is wet AMD.

Embodiment 12. The method of embodiment 9, wherein the neurodegenerative disease is Alzheimer's disease or Parkinson's disease.

Embodiment 13. The method of any one of embodiments 1-12, wherein the nucleic acid further comprises a hVMD2 promoter.

Embodiment 14. The method of any one of embodiments 1-13, wherein the nucleic acid is comprised in a vector.

Embodiment 15. The method of embodiment 14, wherein the vector is an adeno-associated viral (AAV) vector.

Embodiment 16. The method of embodiment 15, wherein the AAV vector is AAV2.

Embodiment 17. The method of any one of embodiments 1-16, wherein the polypeptide comprises the amino acid sequence of SEQ ID NO: 1 except that the serine at one or more of positions 138, 142, 211, 455, 462, 463, 466, 467, and 469 of SEQ ID NO: 1 is, independently, substituted with another amino acid.

Embodiment 18. The method of embodiment 17, wherein the serine at position 138 of SEQ ID NO: 1 is substituted with an alanine.

Embodiment 19. The method of embodiment 17 or 18, wherein the serine at position 142 of SEQ ID NO: 1 is substituted with an alanine.

Embodiment 20. The method of any one of embodiments 17-19, wherein the serine at position 211 of SEQ ID NO: 1 is substituted with an alanine.

Embodiment 21. The method of any one of embodiments 17-20, wherein the serine at position 455 of SEQ ID NO: 1 is substituted with an alanine.

Embodiment 22. The method of any one of embodiments 17-21, wherein the serine at position 462 of SEQ ID NO: 1 is substituted with an aspartic acid.

Embodiment 23. The method of any one of embodiments 17-22, wherein the serine at position 463 of SEQ ID NO: 1 is substituted with an aspartic acid.

Embodiment 24. The method of any one of embodiments 17-23, wherein the serine at position 466 of SEQ ID NO: 1 is substituted with an aspartic acid.

Embodiment 25. The method of any one of embodiments 17-24, wherein the serine at position 467 of SEQ ID NO: 1 is substituted with an alanine.

Embodiment 26. The method of any one of embodiments 17-25, wherein the serine at position 469 of SEQ ID NO: 1 is substituted with an aspartic acid.

Embodiment 27. The method of any one of embodiments 1-26, wherein the subject is a human.

Embodiment 28. The method of any one of embodiments 1-27, wherein the nucleic acid, the polypeptide, or a vector comprising the nucleic acid is administered by subretinal injection, intravitreal injection, or topical administration.

Embodiment 29. A polypeptide comprising the amino acid sequence of SEQ ID NO: 1, except that the serine at each one of positions 138, 142, and 211 of SEQ ID NO: 1 is respectively substituted with an alanine.

Embodiment 30. A polypeptide comprising the amino acid sequence of SEQ ID NO: 1, except that the serine at position 455 of SEQ ID NO: 1 is substituted with an alanine and, optionally, the serine at position 211 of SEQ ID NO: 1 is substituted with an alanine.

Embodiment 31. A polypeptide comprising the amino acid sequence of SEQ ID NO: 1, except that the serine at each one of positions 462, 463, 466, and 469 of SEQ ID NO: 1 is respectively substituted with an aspartic acid and the serine at position 467 of SEQ ID NO: 1 is substituted with an alanine.

Embodiment 32. A nucleic acid encoding the polypeptide of any one of embodiments 29-31.

Embodiment 33. A vector comprising the nucleic acid of embodiment 32.

Embodiment 34. A pharmaceutical composition comprising (a) a pharmaceutically-acceptable carrier and (b) the polypeptide of any one of embodiments 29-31, a nucleic acid encoding the polypeptide, or a vector comprising the nucleic acid.

The following example further illustrate the invention but, of course, should not be construed as in any way limiting its scope.

### EXAMPLE

This example demonstrates that administration of constitutively active TFEB mitigates alterations in lysosomal function and autophagy in RPE cells, thereby inhibiting an AMD-like phenotype.

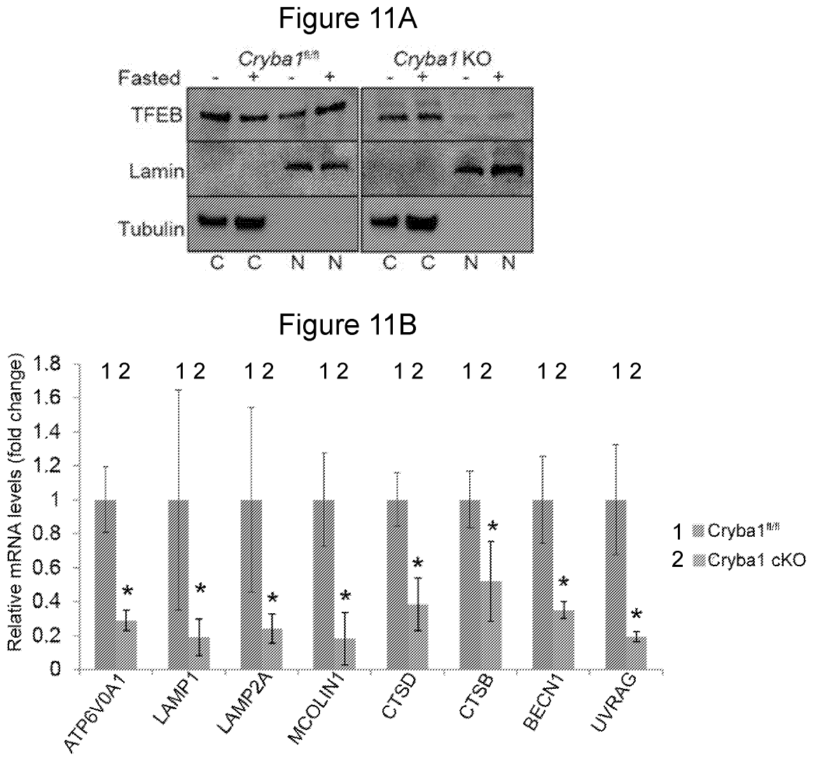

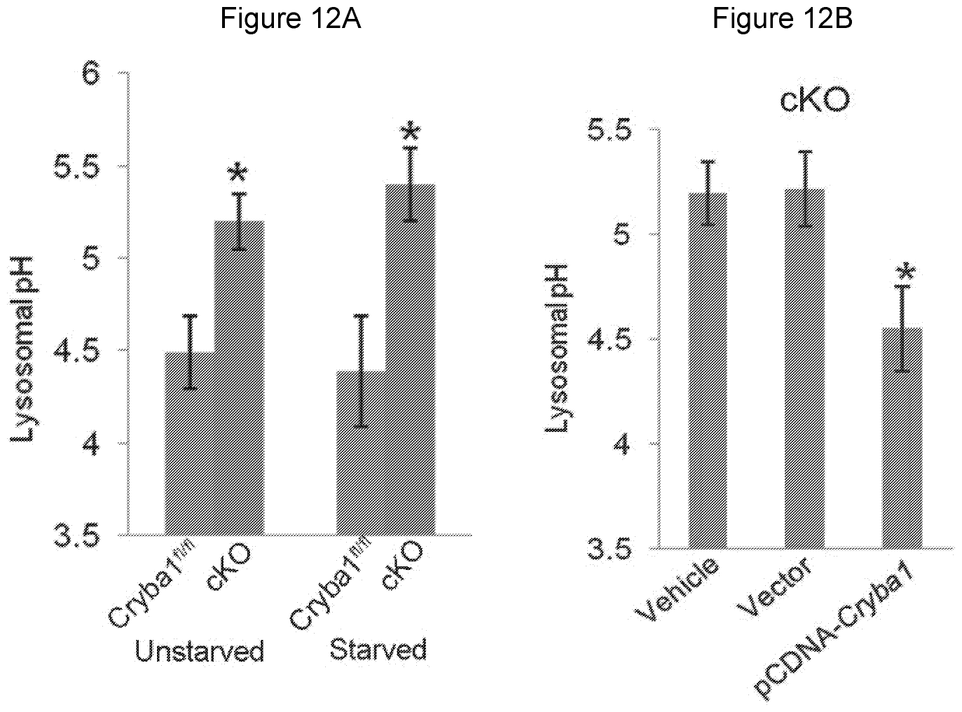

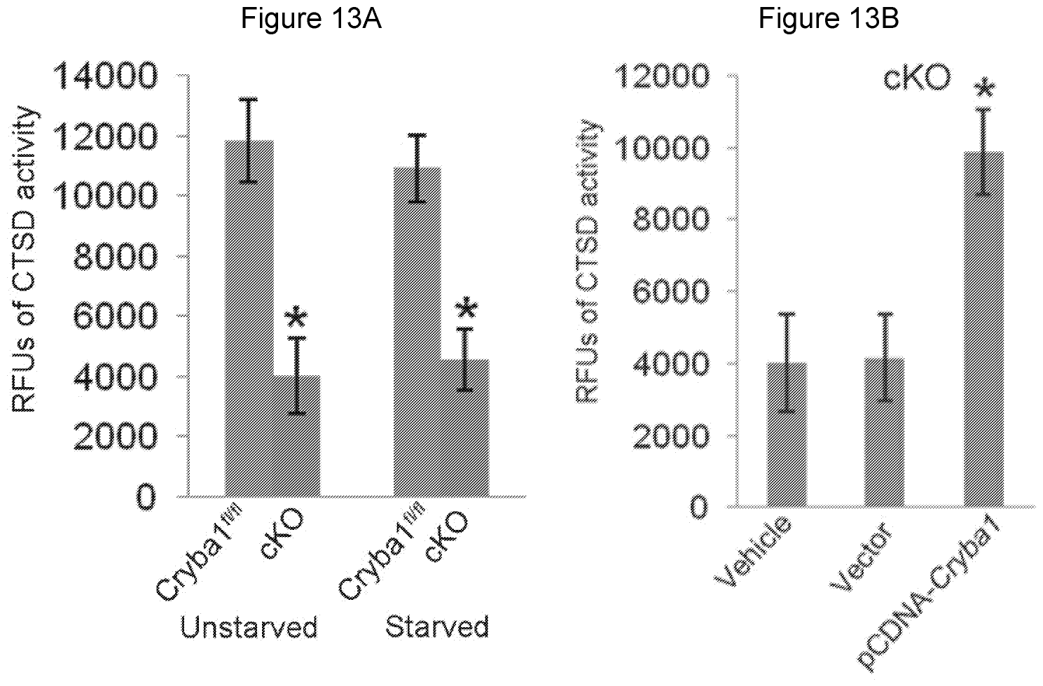

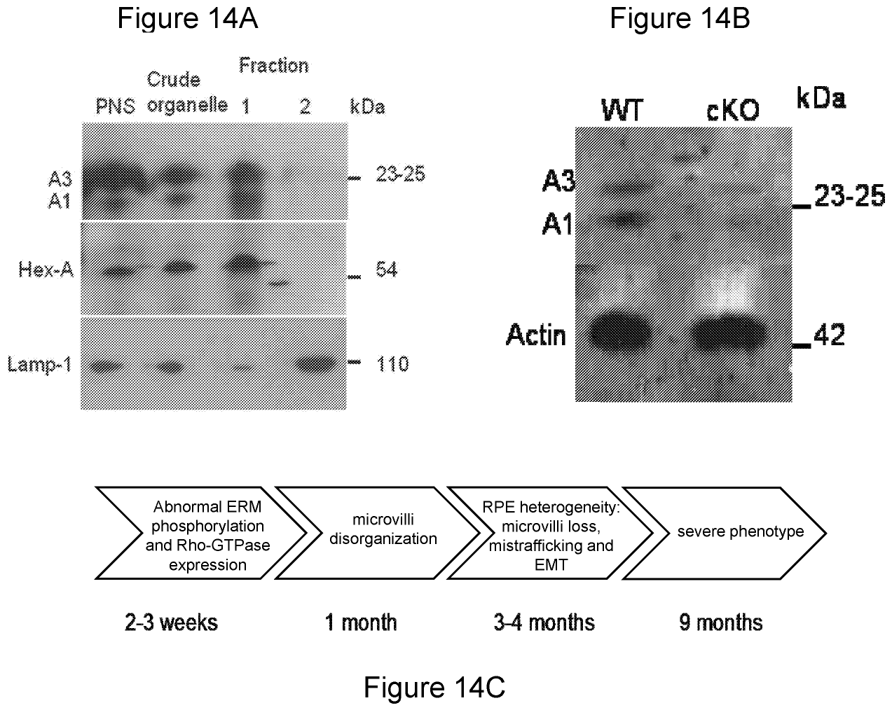

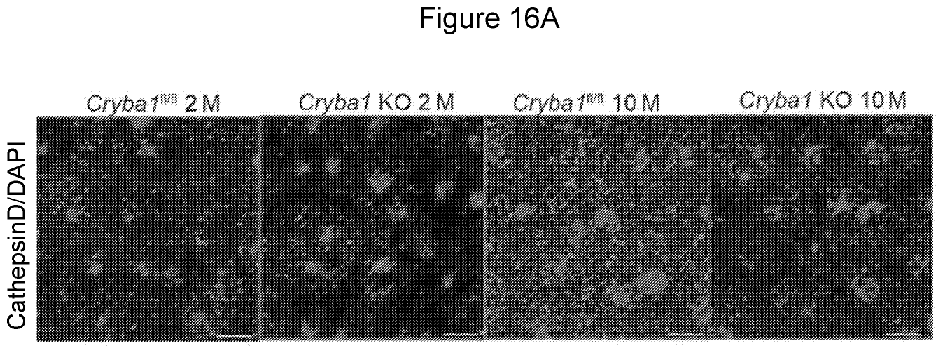

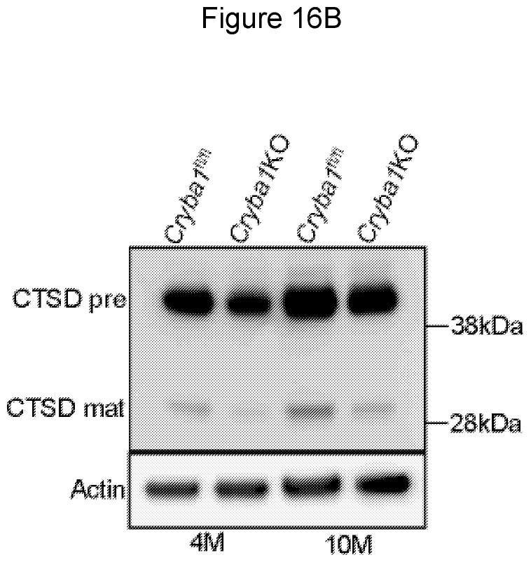

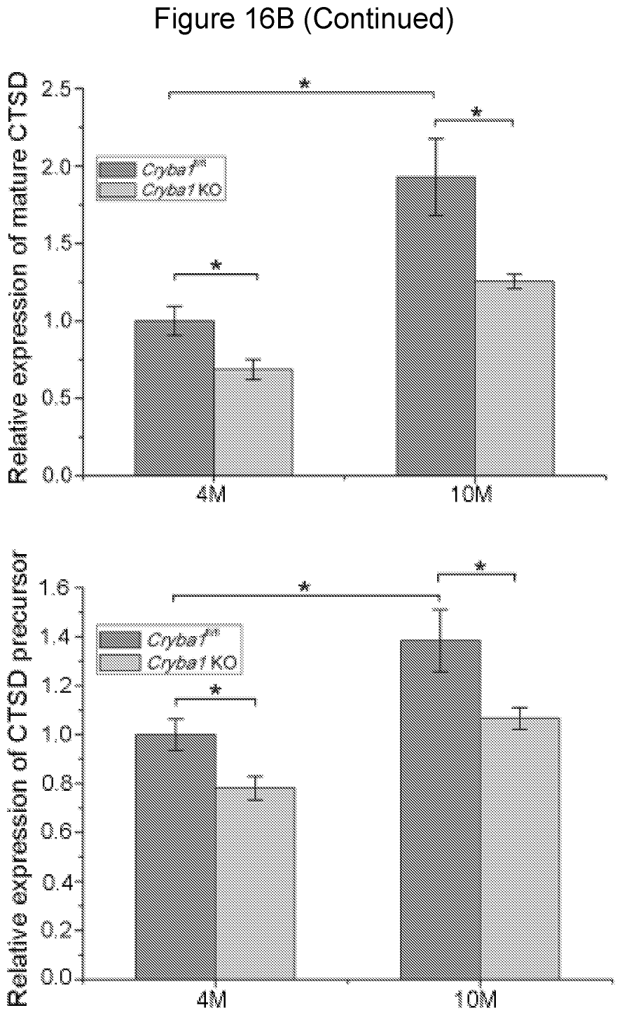

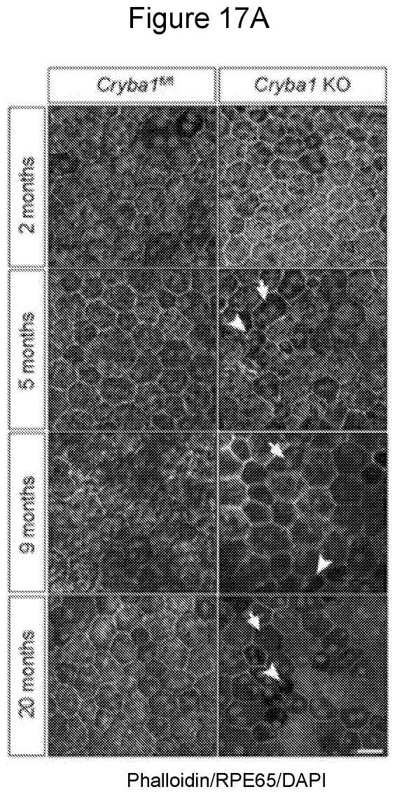

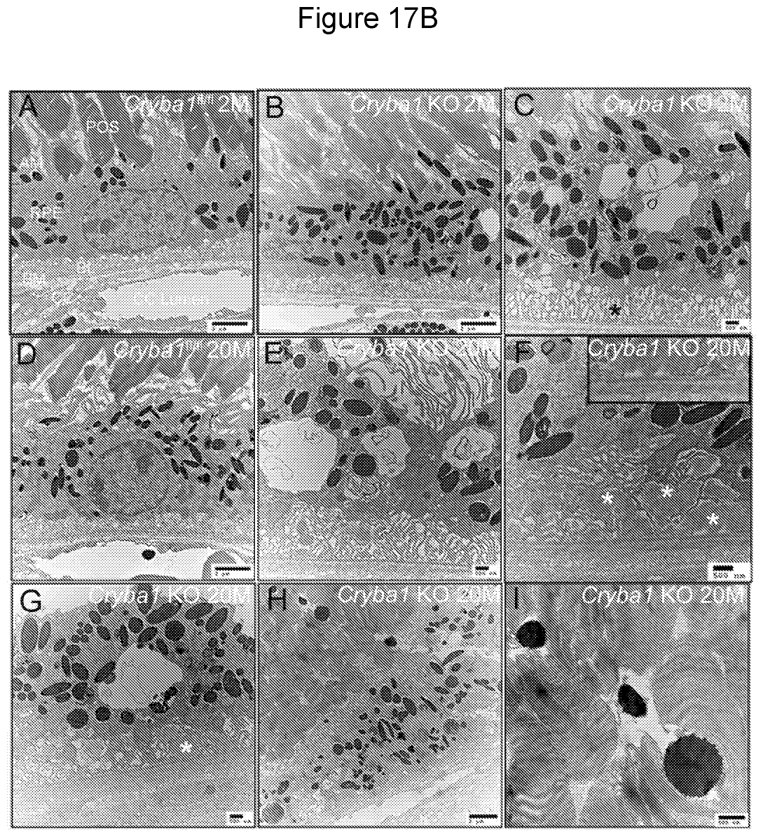

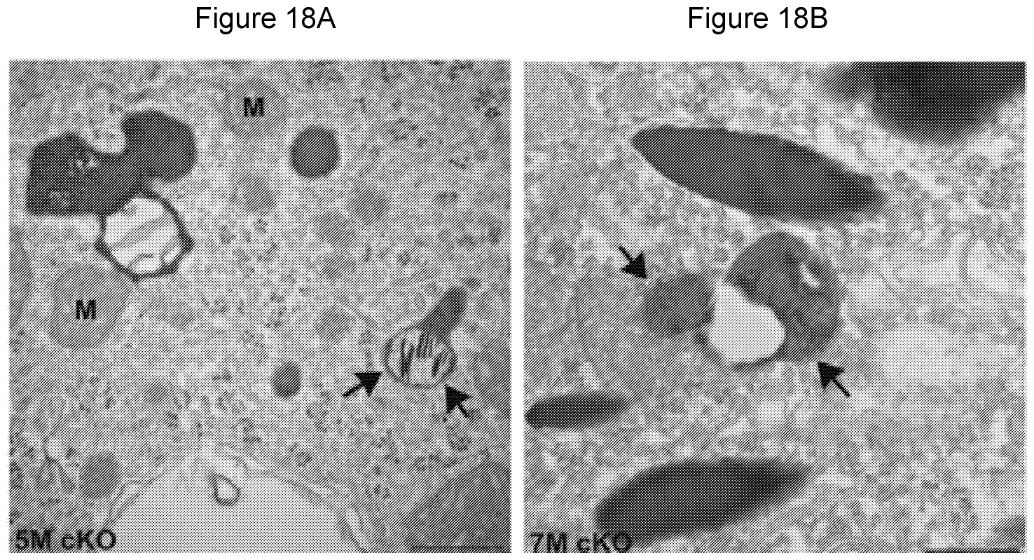

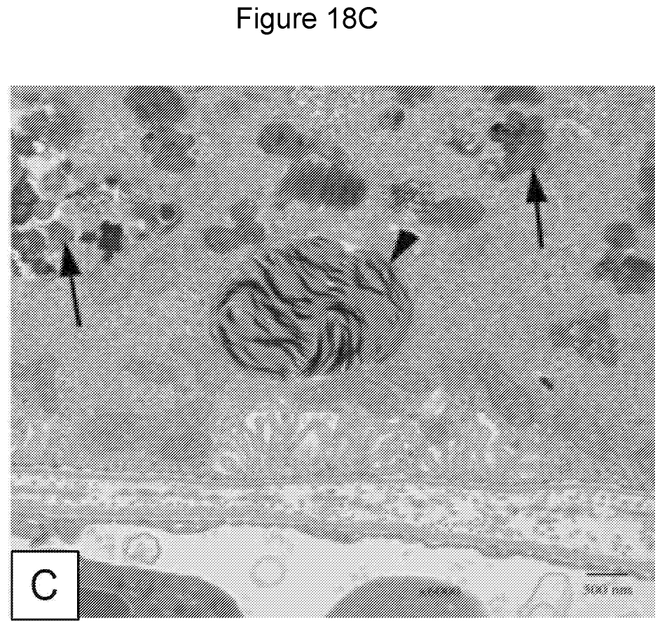

### Cryba1 Knockout and Conditional Knockout Mice (See FIGS. 11-18)

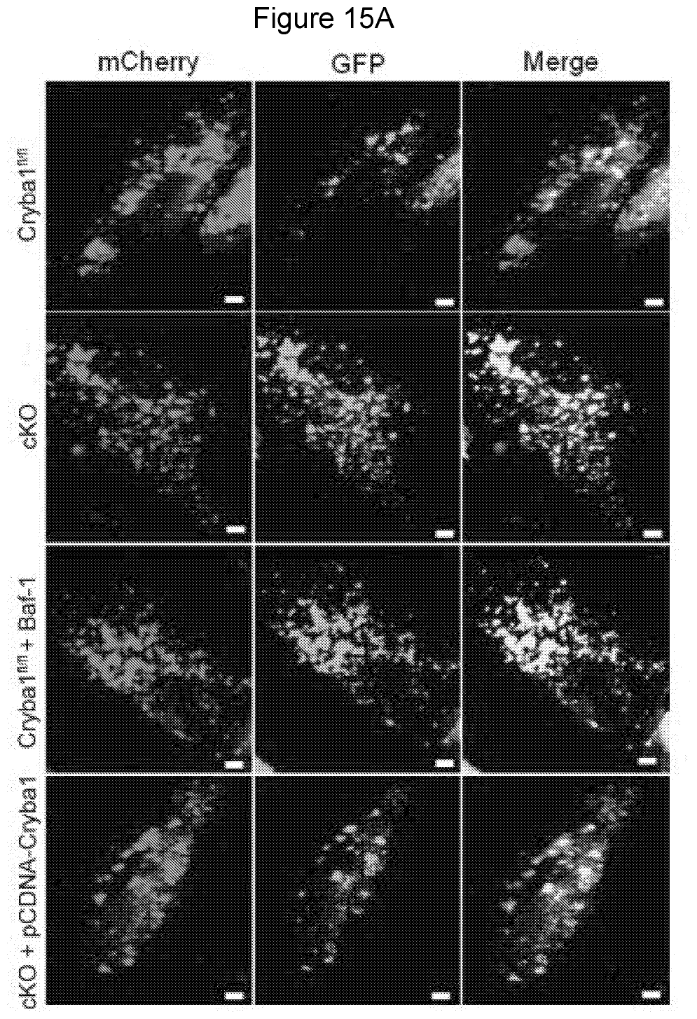

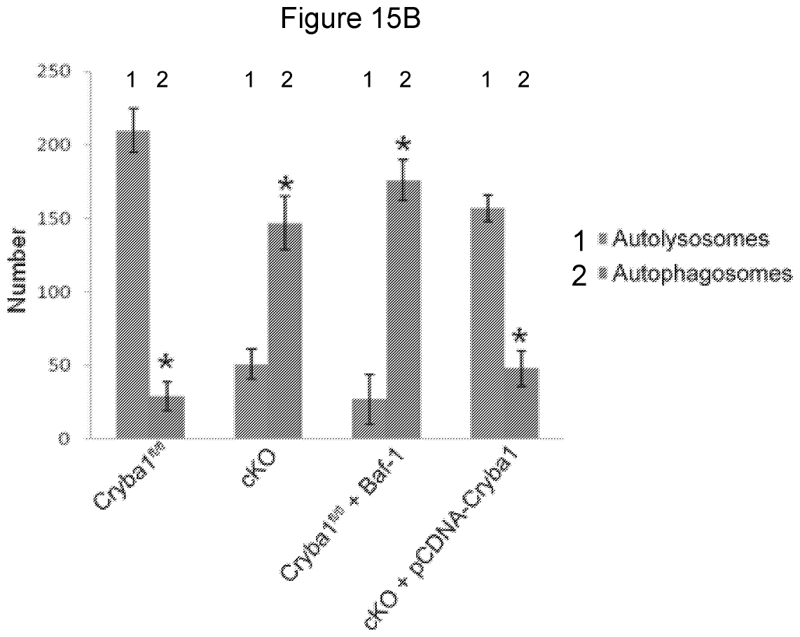

Cryba1 (encoding βA3/A1-crystallin) knockout mice (KO) and conditional knockout (cKO) are a mouse model with AMD-like pathology. βA3/A1-crystallin protein is localized in the lysosomal lumen. In Cryba1 KO or cKO mice, RPE cell abnormalities develop as follows: abnormal ERM phosphorylation and Rho-GTPase expression (2-3 weeks); microvilli disorganization (1 month); RPE heterogeneity, microvilli loss, mistrafficking, and EMT (3-4 months); and severe phenotype (9 months) (see FIG. 15C).

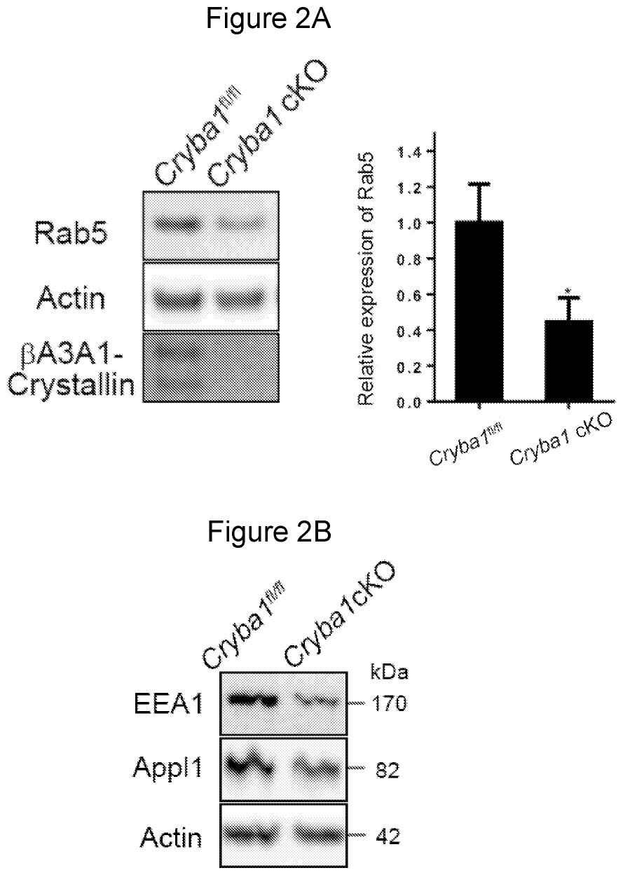

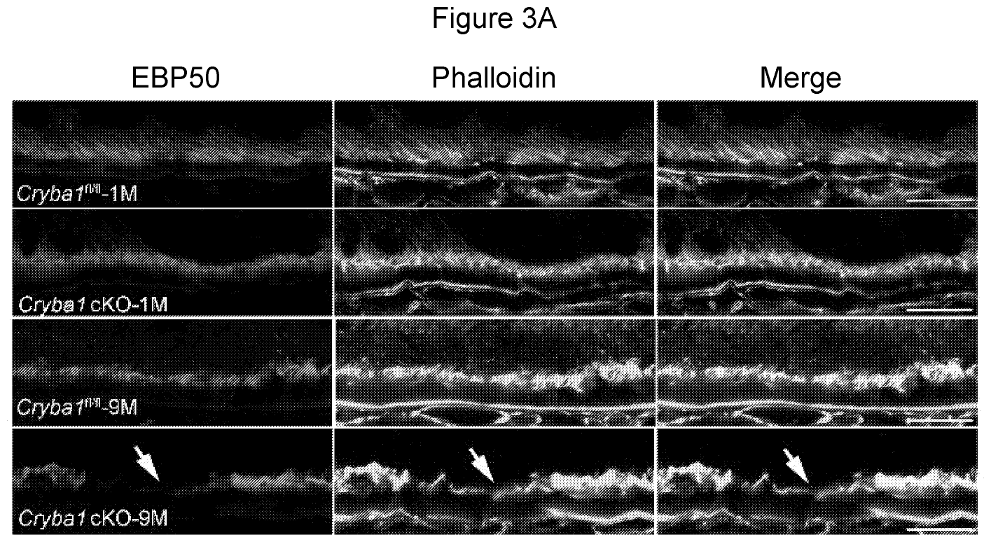

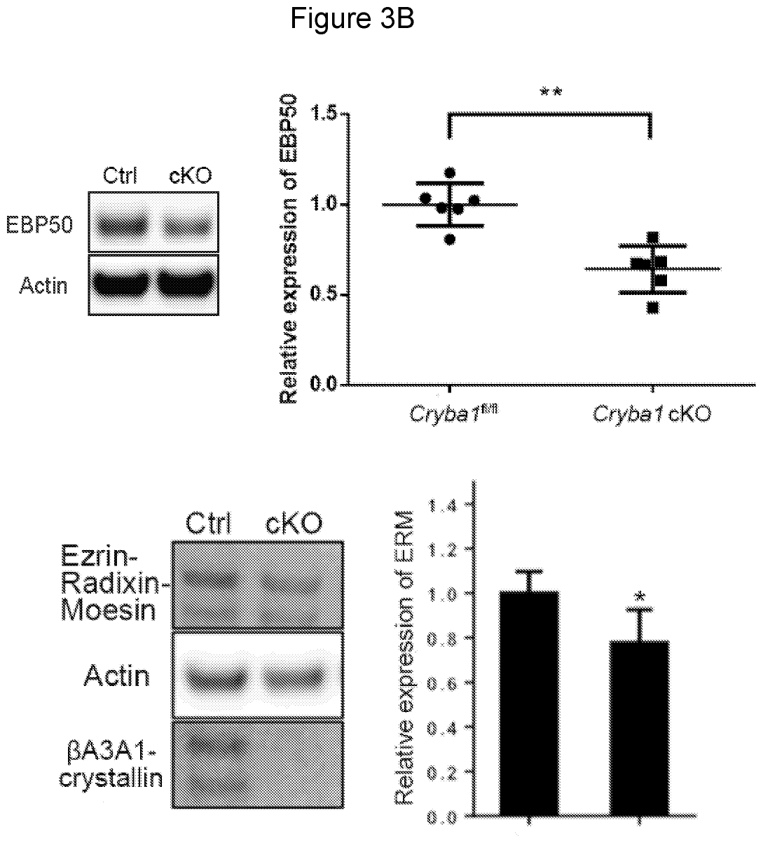

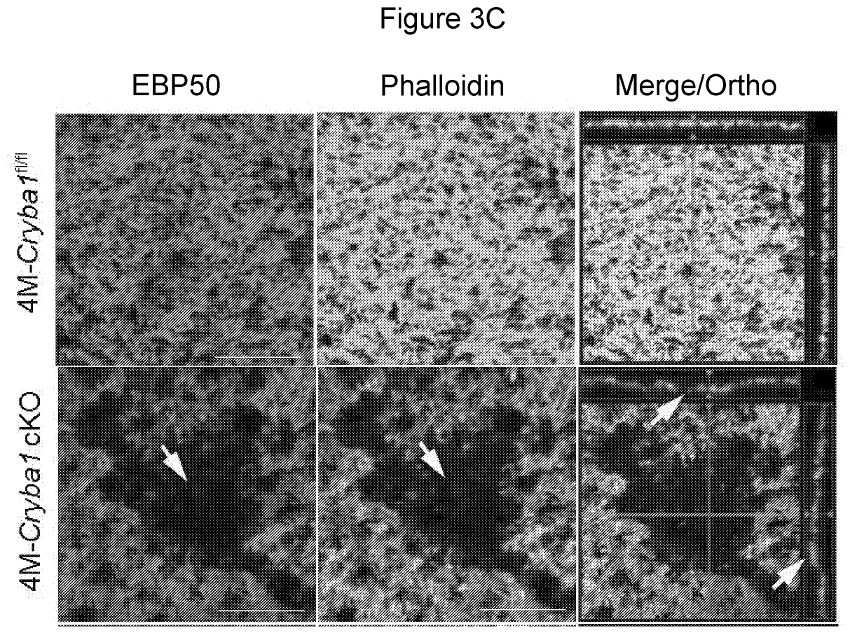

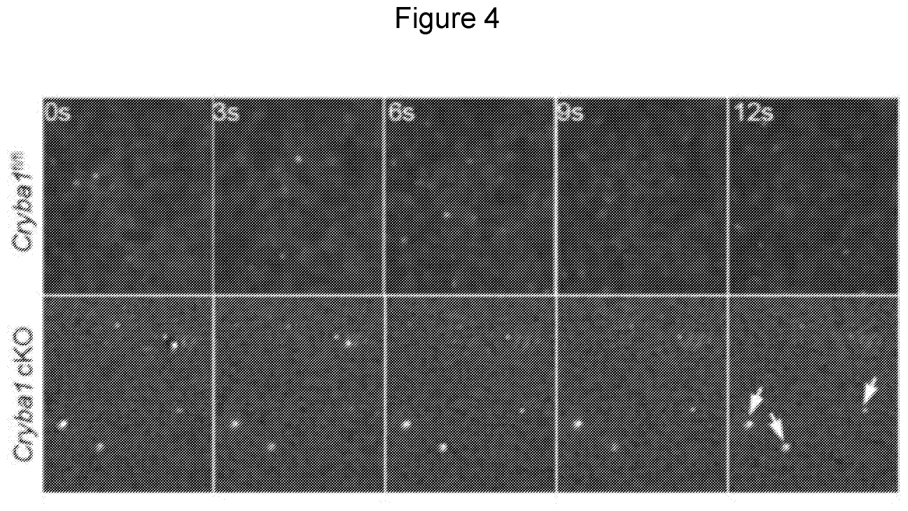

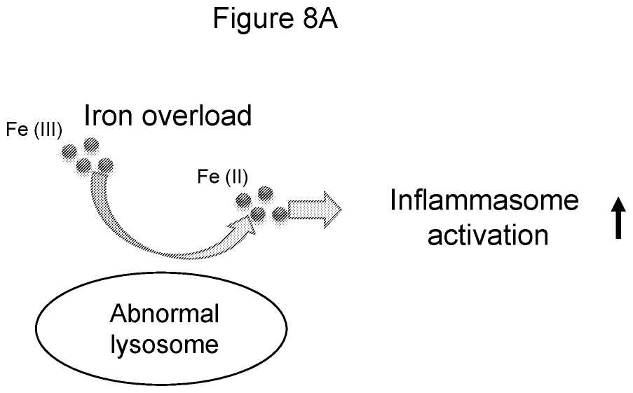

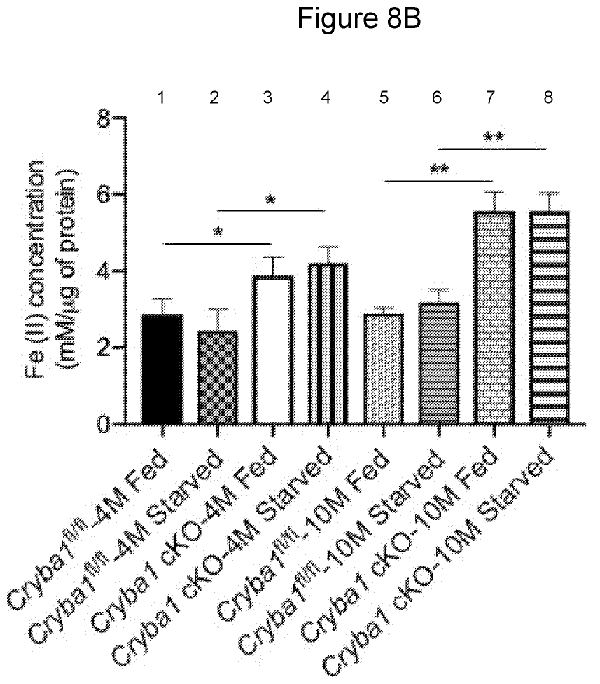

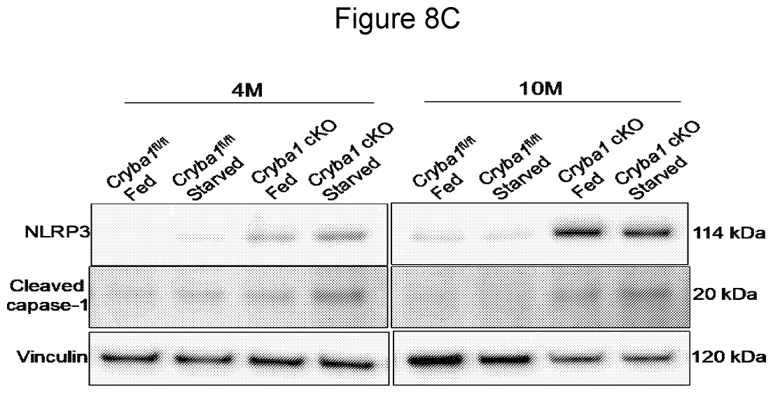

Cryba1 cKO mice have reduced expression of proteins in the endocytic pathway in RPE cells (e.g., Rab5, EEA1, and AAPL1), reduced expression of apical side proteins (e.g., ERM and EBP50) in RPE cells, and altered clathrin-mediated endocytosis in RPE cells (see FIGS. 2A-B, 3A-C, and 4). Cryba1 cKO mice show increased iron accumulation and inflammasome activation in RPE cells (FIGS. 8A-C). Additionally, Cryba1 cKO mice have decreased in expression of CLEAR (coordinated lysosomal expression and regulation) network genes, increased lysosomal pH, decreased cathepsin D enzyme activity, loss of cobblestone-like structure, and decreased expression of RPE65 in RPE cells (see FIGS. 11A-B, 12A-B, 13A-B, 14A-B, and 17A).

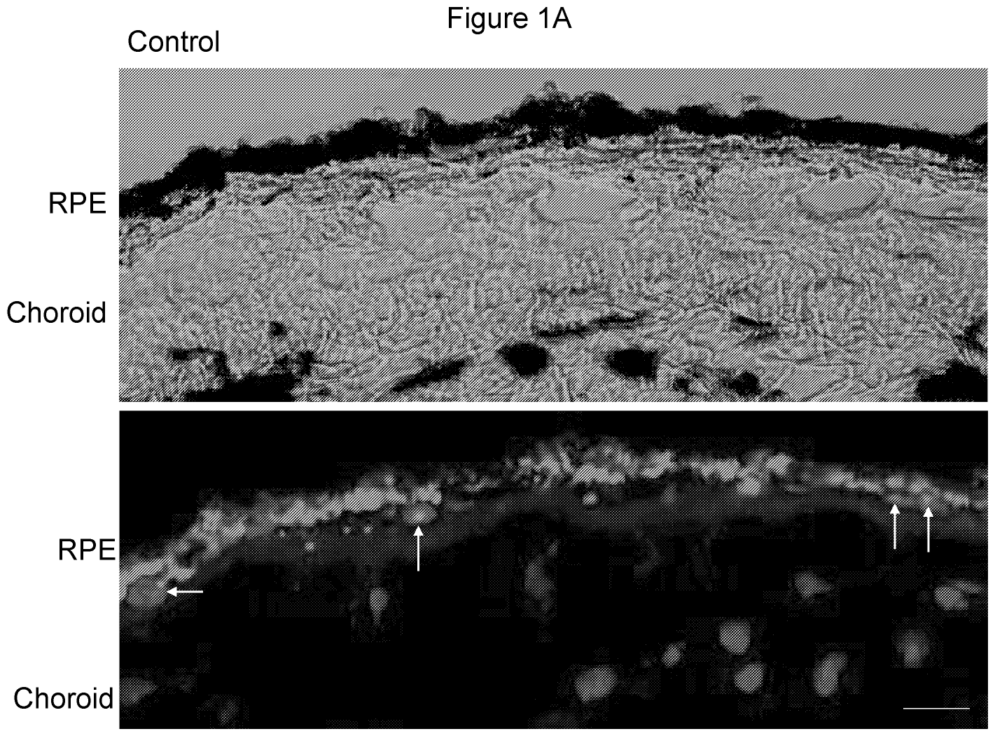

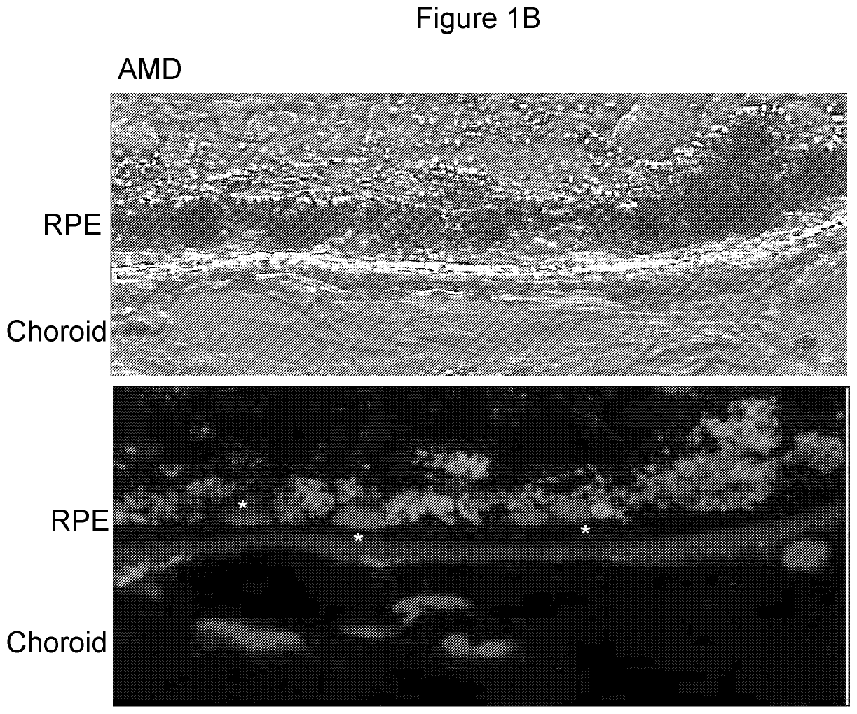

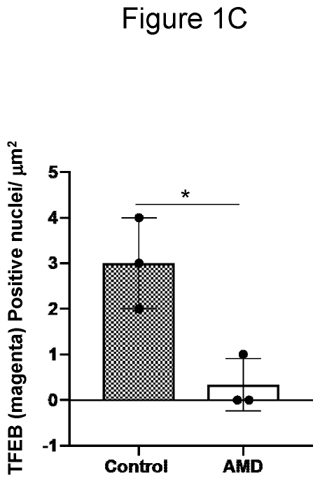

Additionally, TFEB activation is abnormal in RPE cells of human AMD patients and in Cryba1 KO mice (see FIGS. 1A-1C and 11A-11B).

### Production of AAV Vectors Carrying the Wild-Type and Constitutively Active TFEB

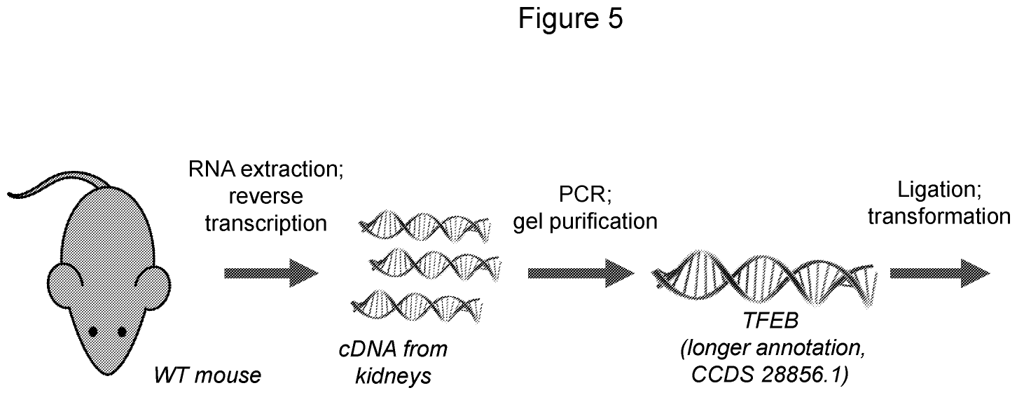

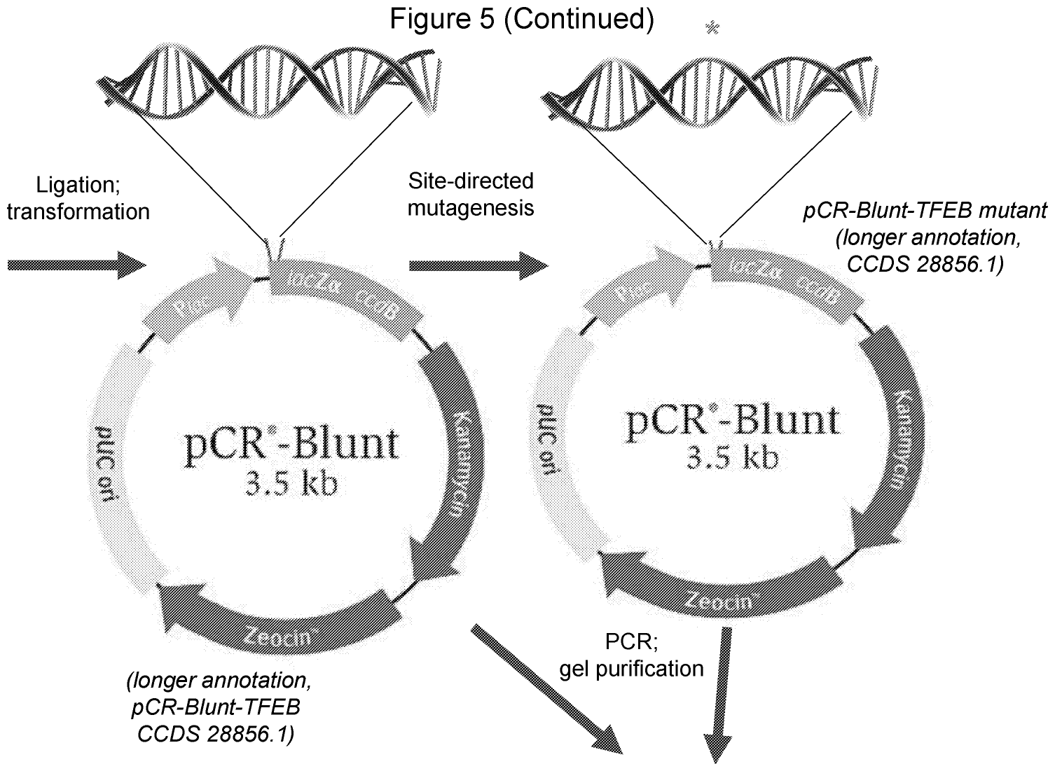

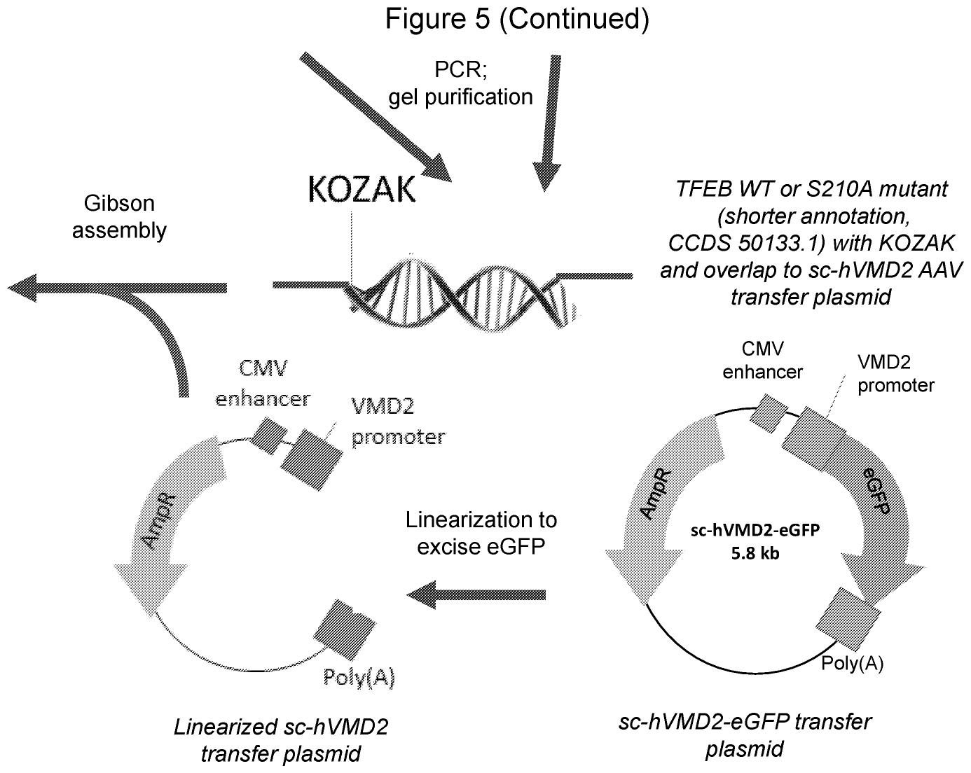

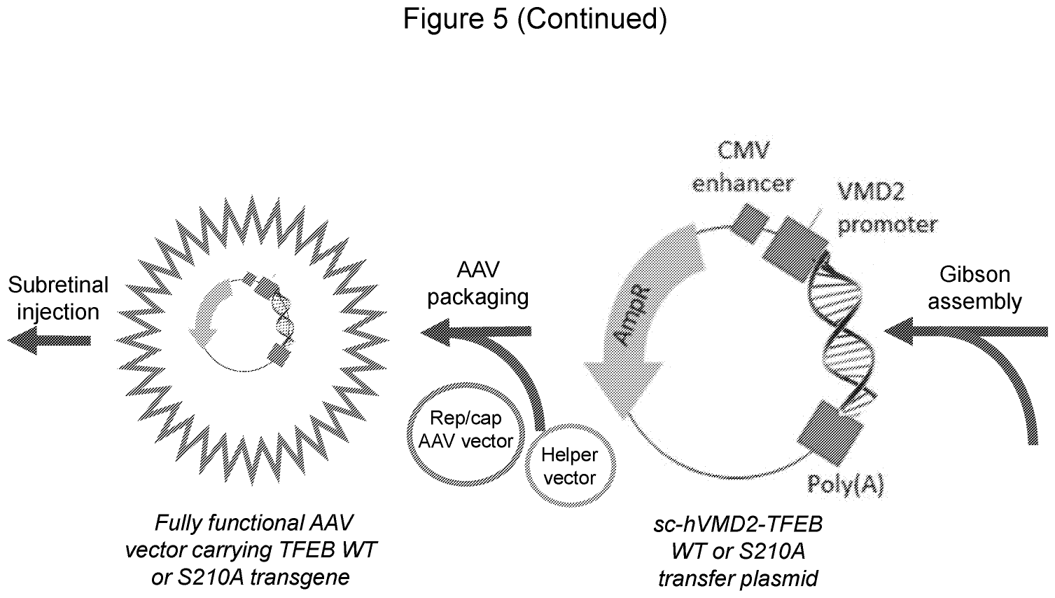

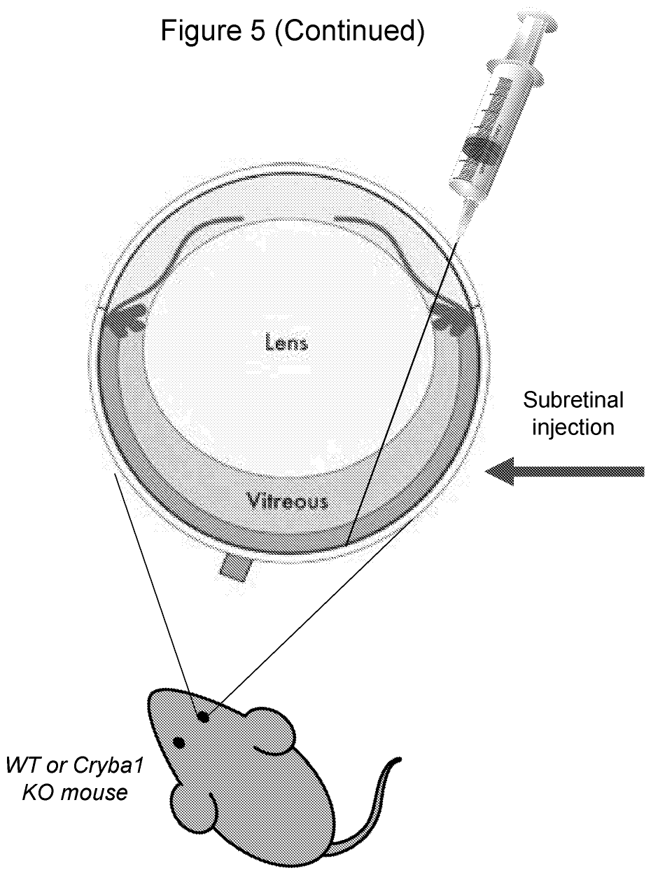

To determine if the AMD-like phenotype of Cryba1 cKO mice could be rescued by constitutively activated TFEB, AAV vectors carrying wild type (WT) and constitutively active TFEB-S210A were engineered as shown in FIG. 5.

WT TFEB was first cloned into a pCR-Blunt vector (Invitrogen, Carlsbad, Calif.). Briefly, RNA was extracted from mouse kidney, followed by reverse transcription to generate cDNA. WT TFEB was amplified by PCR using a set of primers designed for the longer annotation of mouse TFEB (CCDS 28856.1, TFEB_CDS_F: ATGGCGTCACGCATCGGG (SEQ ID NO: 2) and TFEB_CDS_R: TCACAGAACATCACCCTCCT (SEQ ID NO: 3)) and High-Fidelity Phusion polymerase (Thermo Scientific, Waltham, Mass.). The PCR product was gel purified and ligated into the pCR-Blunt vector according to the manufacturer's instructions (confirmed by sequencing).

Alignment of the human and mouse TFEB proteins revealed 93.5% identity in 476 residues overlap corresponding to the shorter annotation of the mouse TFEB (CCDS 50133.1). Notably, in mice there was a deletion of one glutamine residue at the position 44, thus the position of serine critical for the regulation of nuclear localization of TFEB is shifted to position 210 in mice compared to 211 in humans.

To create the TFEB-S210A mutant, the TCC codon was mutated to GCC by introducing a point T->G mutation into the WT TFEB in pCR-Blunt using QuikChange Lightning Site-Directed Mutagenesis Kit (Agilent, Santa Clara, Calif.) according to the manufacturer's instructions (confirmed by sequencing).

To prepare the plasmid for Gibson assembly, eGFP was excised using Agel (BshT1) and Sacl restriction enzymes and the resulting linearized sc (self complementary)-hVMD2 was gel purified. A shorter version of TFEB (CCDS 50133.1) was PCR-amplified from the WT or mutated TFEB in pCR-Blunt vector using the following primers: sc-hVMD2_TFEB_F: accagcctagtcgccagaccaccggGCCACCATGGCGTCACGCATCGGG (SEQ ID NO: 4) and sc-hVMD2_TFEB_R: cacagtcgaggctgatcagcgagctTCACAGAACATCACCCTCCT (SEQ ID NO: 5). Both primers contained sequences overlapping with the linearized sc-hVMD2 transfer plasmid (lower case letters) and the forward primer contained KOZAK sequence GCCACC to improve translation efficacy.

The linearized sc-hVMD2 plasmid and the freshly PCR-amplified WT or TFEB-S210A were ligated using Gibson assembly kit (New England Biolabs, Ipswich, MA). The integrity of the ITRs was confirmed by SmaI digest. The sc-hVMD2-TFEB and sc-hVMD2-TFEB-S210A constructs were packaged into AAV2 and AAV8-2YF vectors using a triple transfection method in AAV-HEK-293 cells (Agilent). Virus was purified on an iodixanol gradient and buffer exchanged into DPBS. Virus was titered using QPCR, and had a titer of 1.64×10¹² vg (vector genomes)/ml (WT) and 1.72×10¹² vg/ml (mutant).

### Transfection of AAV-TFEB Vector into RPE Cells

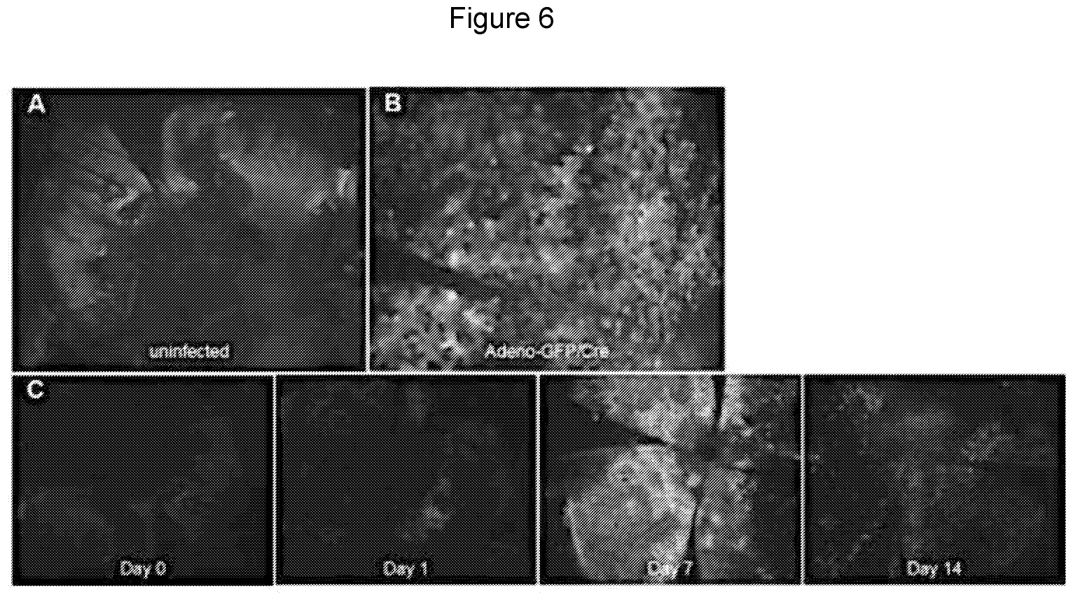

As shown in FIGS. 6A-C, subretinal injection of AAV2-TFEB-GFP demonstrated efficient infection of more than 85-90% of RPE cells after a single subretinal infection. Therefore, the AAV-TFEB vector is suitable for transfection in RPE cells.

### Infection of RPE Cells from Cryba1 KO Mice with AAV Vector Expressing Constitutively Active TFEB Mitigates Alterations in Lysosomal Function and Autophagy

Cryba1 KO mice were prepared as described in Valapala et al., Sci. Rep., 5: 8755 (2015). RPE cells from Cryba1 KO mice were infected with AAV2 vector expressing a constitutively active form of mouse TFEB (TFEB^{S210A}). As a control, RPE cells from Cryba1 KO mice were infected with the AAV vector expressing wild-type mouse TFEB or empty vector.

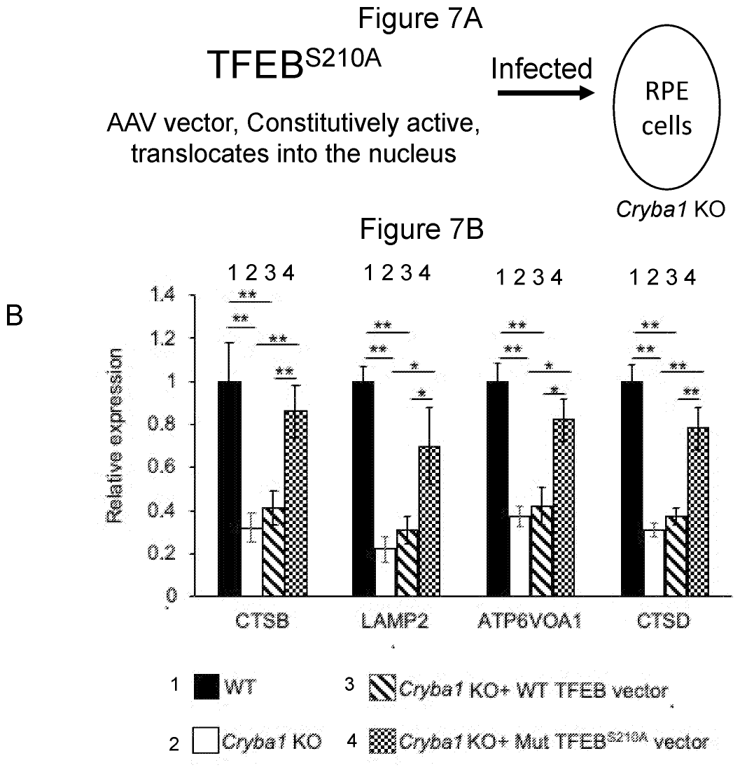

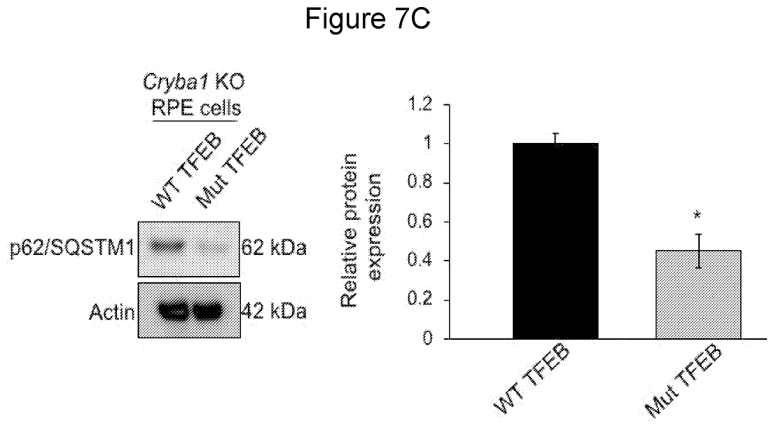

As demonstrated by FIGS. 7A-C and 10A-B, constitutively active TFEB localized to the nucleus of RPE cells of Cryba1 KO and cKO mice and reduced alterations in CLEAR network gene expression and autophagosome accumulation, respectively.

### Infection of RPE Cells from Cryba1 KO Mice with AAV Vector Expressing Constitutively Active TFEB Reduces Iron Accumulation and Inflammasome Activation

As demonstrated by FIGS. 8A-C, total iron and redox sensitive ferrous ion levels were increased in RPE cells of aged Cryba1 cKO mice as compared to controls. Furthermore, levels of inflammasome markers NLRP3 and IL-1β were elevated in RPE cells of aged Cryb1 cKO mice relative to age-matched controls. Iron overload due to lysosomal abnormality in RPE cells can cause inflammasome activation and cell death.

To ascertain whether constitutively active TFEB can rescue this phenotype, RPE cells from Cryba1 KO mice were infected with the AAV vector expressing a constitutively active form of mouse TFEB (TFEB^{S210A}) in the presence of iron chelator LCN-2 and an iron source FAC. As a control, RPE cells from Cryba1 KO mice were infected with the AAV vector expressing wild-type mouse TFEB in the presence of iron chelator LCN-2 and an iron source FAC.

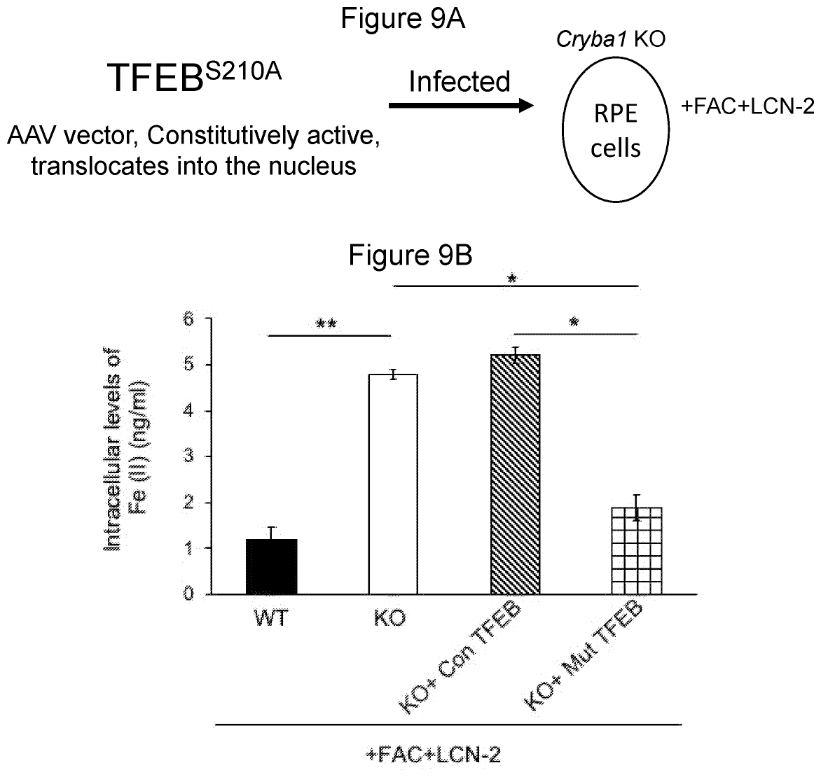

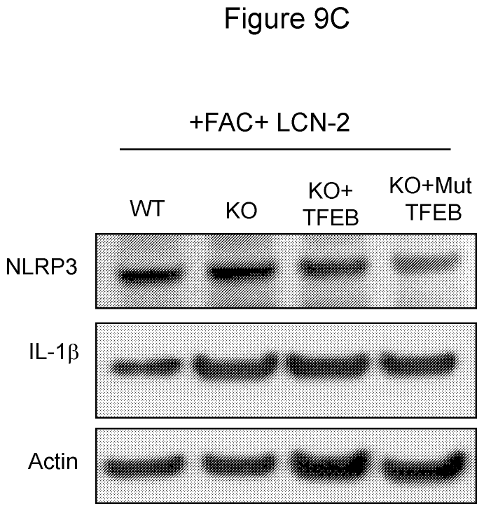

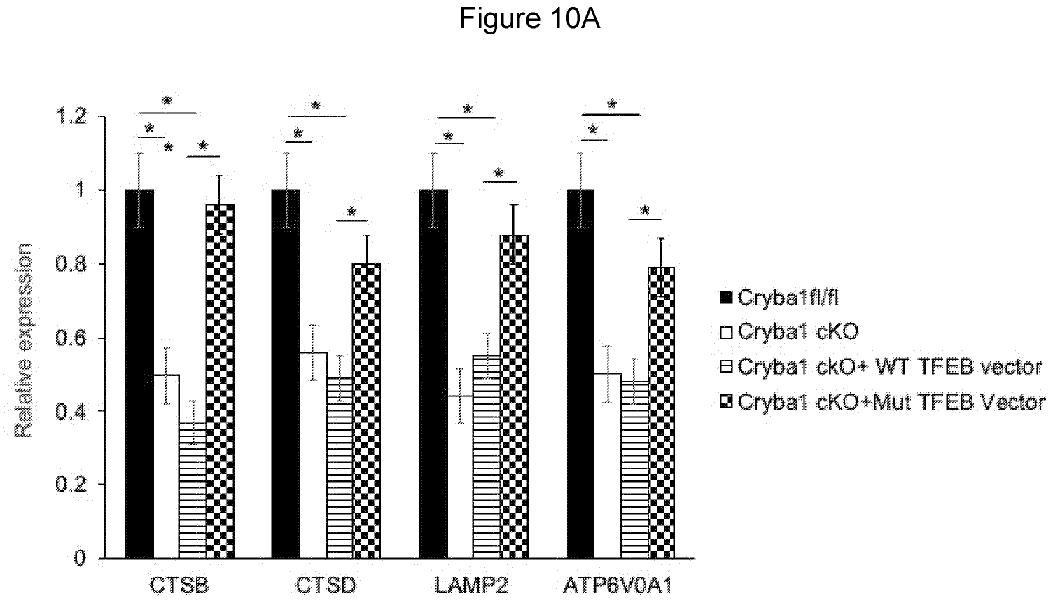

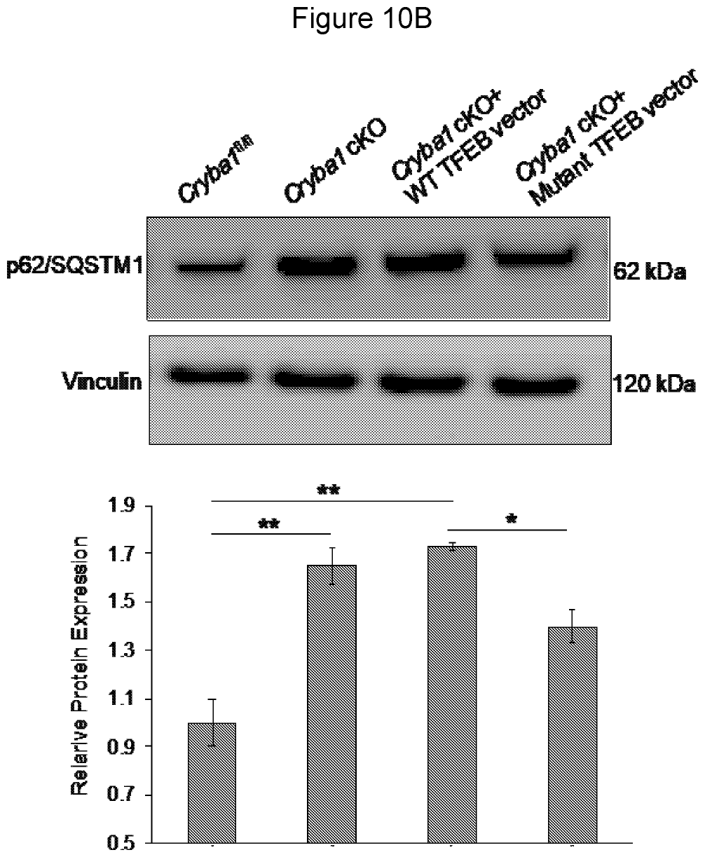

Iron accumulation and inflammasome activation (increased NKRP3 and IL-1β expression) were reversed in mutant TFEB vector-infected Cryba1 KO RPE cells compared to control (WT) TFEB vector-infected cells (see FIGS. 9A-C).

As such, the administration of constitutively active TFEB mitigates alterations in lysosomal function and autophagy in RPE cells, which supports the use of constitutively active TFEB in the treatment or prevention of disorders, such as AMD, diabetic retinopathy, and diabetic macular edema, and neurodegenerative diseases, such as Alzheimer's disease or Parkinson's disease.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of restoring lysosomal function of retinal pigment epithelial (RPE) cells comprising administering (i) a nucleic acid encoding a polypeptide comprising a constitutively active form of transcription factor EB (TFEB) or (ii) the polypeptide to a subject in need thereof.

2. The method of claim 1, wherein the subject has age-related macular degeneration (AMD) or is at risk for developing AMD.

3. The method of claim 2, wherein the AMD is atrophic AMD, optionally wherein the atrophic AMD includes geographic atrophy.

4. The method of claim 2, wherein the AMD is wet AMD.

5. The method of claim 1, wherein the subject has Stargardt's macular retinal degeneration or is at risk for developing Stargardt's macular retinal degeneration.

6. The method of claim 1, wherein the subject has a neurodegenerative disease or is at risk for developing a neurodegenerative disease.

7. The method of claim 6, wherein the neurodegenerative disease is Alzheimer's disease or Parkinson's disease.

8. The method of claim 1, wherein the subject has diabetic retinopathy (DR) or is at risk for developing DR.

9. A method of preventing and/or treating age-related macular degeneration (AMD), Stargardt's macular retinal degeneration, neurodegenerative disease, or diabetic retinopathy in a subject comprising administering (i) a nucleic acid encoding a polypeptide comprising a constitutively active form of transcription factor EB (TFEB) or (ii) the polypeptide to the subject.

10. The method of claim 9, wherein the AMD is atrophic AMD, optionally wherein the atrophic AMD includes geographic atrophy.

11. The method of claim 9, wherein the AMD is wet AMD.

12. The method of claim 9, wherein the neurodegenerative disease is Alzheimer's disease or Parkinson's disease.

13. The method of claim 1, wherein the nucleic acid further comprises a hVMD2 promoter.

14. The method of claim 1, wherein the nucleic acid is comprised in a vector.

15. The method of claim 14, wherein the vector is an adeno-associated viral (AAV) vector.

16. The method of claim 15, wherein the AAV vector is AAV2.

17. The method of claim 1, wherein the polypeptide comprises the amino acid sequence of SEQ ID NO: 1 except that the serine at one or more of positions 138, 142, 211, 455, 462, 463, 466, 467, and 469 of SEQ ID NO: 1 is, independently, substituted with another amino acid.

18. The method of claim 17, wherein:
(a) the serine at position 138 of SEQ ID NO: 1 is substituted with an alanine;
(b) the serine at position 142 of SEQ ID NO: 1 is substituted with an alanine;
(c) the serine at position 211 of SEQ ID NO: 1 is substituted with an alanine;
(d) the serine at position 455 of SEQ ID NO: 1 is substituted with an alanine;
(e) the serine at position 462 of SEQ ID NO: 1 is substituted with an aspartic acid;
(f) the serine at position 463 of SEQ ID NO: 1 is substituted with an aspartic acid;
(g) the serine at position 466 of SEQ ID NO: 1 is substituted with an aspartic acid;
(h) the serine at position 467 of SEQ ID NO: 1 is substituted with an alanine; and/or
(i) the serine at position 469 of SEQ ID NO: 1 is substituted with an aspartic acid.

19-26. (canceled)

27. The method of claim 1, wherein the subject is a human.

28. The method of claim 1, wherein the nucleic acid, the polypeptide, or a vector comprising the nucleic acid is administered by subretinal injection, intravitreal injection, or topical administration.

29. A polypeptide comprising the amino acid sequence of SEQ ID NO: 1, except that:
(a) the serine at each one of positions 138, 142, and 211 of SEQ ID NO: 1 is respectively substituted with an alanine;
(b) the serine at position 455 of SEQ ID NO: 1 is substituted with an alanine and, optionally, the serine at position 211 of SEQ ID NO: 1 is substituted with an alanine; or
(c) the serine at each one of positions 462, 463, 466, and 469 of SEQ ID NO: 1 is respectively substituted with an aspartic acid and the serine at position 467 of SEQ ID NO: 1 is substituted with an alanine.

30-31. (canceled)

32. A nucleic acid encoding the polypeptide of claim 29 or a vector comprising the nucleic acid.

33. (canceled)

34. A pharmaceutical composition comprising (a) a pharmaceutically-acceptable carrier and (b) the polypeptide of claim 29, a nucleic acid encoding the polypeptide, or a vector comprising the nucleic acid.


SEQUENCE LISTING (5 sequences)

SEQ ID NO: 1 - protein, 476 aa, Artificial Sequence
  Synthetic
MASRIGLRMQLMREQAQQEEQRERMQQQAVMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVL

SEQ ID NO: 2 - DNA, 18 bp, Artificial Sequence
  Synthetic
atggcgtcacgcatcggg

SEQ ID NO: 3 - DNA, 20 bp, Artificial Sequence
  Synthetic
tcacagaacatcaccctcct

SEQ ID NO: 4 - DNA, 49 bp, Artificial Sequence
  Synthetic
accagcctagtcgccagaccaccgggccaccatggcgtcacgcatcggg

SEQ ID NO: 5 - DNA, 45 bp, Artificial Sequence
  Synthetic
cacagtcgaggctgatcagcgagcttcacagaacatcaccctcct